(19) United States
(12) Patent Application Publication
(10) Pub. No.: US 2025/0325513 A1
(43) Pub. Date: Oct. 23, 2025

(54) CABAZITAXEL PRODRUG ANTI-TUMOR PREPARATION

(71) Applicant: SHENYANG PHARMACEUTICAL UNIVERSITY, Shenyang, Liaoning (CN)

(72) Inventors: Jin SUN, Shenyang, Liaoning (CN); Bingjun SUN, Shenyang, Liaoning (CN); Zhonggui HE, Shenyang, Liaoning (CN); Yu ZHANG, Shenyang, Liaoning (CN); Danping WANG, Shenyang, Liaoning (CN); Cong LUO, Shenyang, Liaoning (CN); Shiyi ZUO, Shenyang, Liaoning (CN); Lingxiao LI, Shenyang, Liaoning (CN); Chaoying DU, Shenyang, Liaoning (CN)

(21) Appl. No.: 18/998,004

(22) PCT Filed: Dec. 6, 2022
(86) PCT No.: PCT/CN2022/136743

§ 371 Date: Jan. 23, 2025

(30) Foreign Application Priority Data
Dec. 15, 2021 (CN) 202111532693.5

(51) Int. Cl. A61K 31/337; A61K 9/51; A61P 35/00

(52) U.S. Cl. CPC A61K 31/337; A61K 9/5123; A61K 9/5192; A61P 35/00

(57) ABSTRACT

The present invention relates to a cabazitaxel prodrug anti-tumor preparation, designs and synthesizes a small molecule cabazitaxel prodrug with branched fatty alcohol involving formulas (I), (II) and (III) and containing different fatty alcohol side chains and different linking chains, and prepares a self-assembled nanoparticle. Results showed that the self-assembled nanoparticle of the small molecule cabazitaxel prodrug with branched fatty alcohol can effectively improve the efficacy of cabazitaxel, reduce toxic and side effects. The length of branched fatty alcohol side chains, the structure of the fatty alcohol side chains, the elemental composition of the linking chains and the length of the linking chains significantly affect preparation properties, in vivo fate and anti-tumor activity of the cabazitaxel-branched fatty alcohol prodrug self-assembled nanoparticle, which exhibits higher anti-tumor activity and lower toxicity compared with the self-assembled nanoparticle of small molecule cabazitaxel prodrug with straight-chain fatty alcohol.

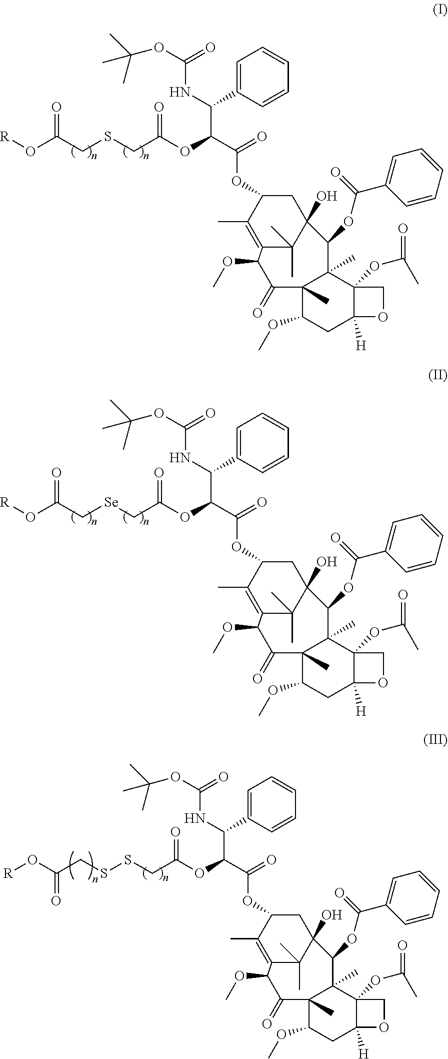

### FIELD OF THE INVENTION

The present invention relates to the field of new excipients and new dosage forms for pharmaceutical preparations, and relates to a cabazitaxel prodrug anti-tumor preparation, and in particular to construction of a cabazitaxel-branched fatty alcohol prodrug and a self-assembled nanoparticle thereof, and application thereof to a drug delivery system.

### THE PRIOR ARTS

In recent years, the incidence of malignant tumors has been on the rise, posing a significant threat to human health. Chemotherapy remains one of the most effective strategies in cancer treatment. Cabazitaxel (CTX), the second-general taxane compound, is applied as therapeutic drugs for chemotherapy and exhibits potent cytotoxicity and anti-tumor effects. However, cabazitaxel can induce severe adverse reactions, including gastrointestinal disturbances, allergic reactions, renal failure, and neutropenia. Moreover, cabazitaxel is poorly water soluble, and the marketed formulation (Jevtana®, Sanofi-Aventis) uses a large number of solubilization agents (polysorbate 80 and ethanol) to improve the solubility of CTX. Even with the aid of solubilizers, cabazitaxel solutions exhibit poor stability, tend to precipitate after dilution, and possess suboptimal pharmacokinetic properties. These drawbacks significantly limit the clinical application of cabazitaxel.

Prodrug strategies are an effective way to improve the delivery efficiency of chemotherapeutic drugs. Implementing structural modifications on cabazitaxel via prodrug strategies can effectively address the issues of poor solubility and significant toxic side effects associated with cabazitaxel. Nanodrug delivery system can effectively prolong the circulation time of drugs in bodies and enhance anti-tumor effects. Thus, the prodrug-based self-assembled nanodrug delivery system integrates the advantages of prodrug strategies and nanotechnology, has the advantages of being high in drug loading capacity, free from solubilizers, etc., and thus has been widely studied in recent years.

Prodrugs usually consist of three parts: parent drug, linking chain, and side chain. The parent drug and the side chain are linked together by the linking chain. In order to construct the prodrugs having self-assembly capability, most of existing cabazitaxel prodrugs use fatty acids or fatty alcohols with a straight-chain structure as side chains. Aliphatic side chains can improve the structural flexibility of prodrug molecules, balance intermolecular interaction, and promote the self-assembly of the prodrugs. We hypothesize that branched fatty alcohols can effectively disrupt the tight packing of the prodrug molecules, which is expected to further enhance the self-assembly capability of the prodrugs. In addition, the carbon chain length of the branched fatty alcohols may affect the pharmaceutical properties, in vivo fate, and anti-tumor effects of prodrug-based self-assembled nanoparticles. There are no studies comparing the effect of the length of carbon chains of the branched fatty alcohols on the self-assembled nanoparticle of the prodrug, and no studies have reported the effect of the branched-chain fatty alcohols and straight-chain fatty alcohols as side chains on the prodrug-based self-assembled nanoparticles.

The tumor microenvironment is significantly different from the microenvironment of normal tissue cells. Large quantities of reactive oxygen species and glutathione are produced in tumor cells, resulting in a tumor microenvironment with an imbalanced redox state. Thioether bonds, disulfide bonds, and selenoether bonds all have dual redox-sensitive properties, which can respond to the high redox state in the tumor cells and further intelligently release drugs. Various linking chains exhibit distinct elemental compositions and different sensitivities to redox reactions. Thus, cabazitaxel prodrugs modified with different linking chains exhibit distinct characteristics in terms of pharmaceutical properties, in vivo behavior, and anti-tumor efficacy. In addition, the chain length of the linking chain also affects the redox sensitivity of the prodrug-based self-assembled nanoparticles, which in turn affects the anti-tumor activity of the prodrug-based self-assembled nanoparticles.

### SUMMARY OF THE INVENTION

The present invention is designed to overcome the defects existing in the prior art by providing a cabazitaxel prodrug anti-tumor preparation including particularly a series of small molecule cabazitaxel prodrugs with branched fatty alcohols, a kind of nanoparticles thereof, and their preparation and application. The nanoparticle is a self-assembly nanoparticle with the advantages of small particle size, uniform distribution, high drug loading capacity, excellent stability, potent anti-tumor effects, and good safety.

The present invention aims to design and synthesize a series of cabazitaxel prodrugs containing branched fatty alcohol side chains of different lengths, straight-chain fatty alcohol side chains of different lengths, and different linking chains, and to prepare the self-assembly nanoparticles. Experimental results showed that the length of the branched fatty alcohol side chains, the structure of the (branched or straight-chain) fatty alcohol side chains, elemental composition of the linking chains, and the length of the linking chains affect the anti-tumor effects and safety of the prodrug self-assembly nanoparticles. The present invention provides more options for the development of a novel prodrug self-assembly nanodrug delivery system and meets urgent clinical needs for highly effective-low toxicity chemotherapy preparations.

In order to realize the above purposes, the present invention adopts the following technical solutions:

A series of small-molecule cabazitaxel prodrugs with branched fatty alcohols or their pharmaceutically acceptable salts thereof, where the general structures of the cabazitaxel-branched fatty alcohol small-molecule prodrug are as shown in following formulas (I), (II) and (III):

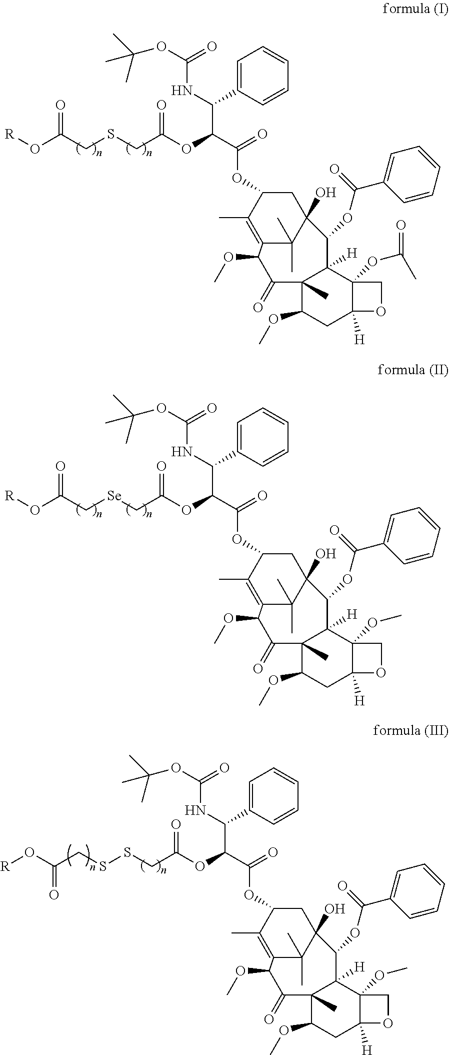

where n=1-3;
R can be either a saturated or unsaturated C₃-C₃₀ hydrocarbon group. R is a hydrocarbon group containing a branched structure and the branched chain is one or more of a C₁-C₁₈ alkyl group, a C₂-C₁₈ alkenyl group, or a C₂-C₁₈ alkynyl group.

Further, R can be either a saturated or unsaturated C₃-C₂₄ hydrocarbon group. R is a hydrocarbon group containing a branched structure and the branched chain is one or more of a straight-chain C₆-C₁₀ alkyl group, a straight-chain C₆-C₁₀ alkenyl group, or a straight-chain C₆-C₁₀ alkynyl group.

Further, R can be either a saturated or unsaturated C₁₀-C₂₄ hydrocarbon group. R is a hydrocarbon group containing a branched structure and the branched chain is one or more of a straight-chain C₆-C₁₀ alkyl group, a straight-chain C₆-C₁₀ alkenyl group, or a straight-chain C₆-C₁₀ alkynyl group.

Further, R is a C₁₀-C₂₄ hydrocarbon group. R is a hydrocarbon group containing a branched structure and the branched chain is a straight-chain C₆-C₁₀ alkyl group.

Further, R can be either a saturated or unsaturated C₁₆-C₂₄ hydrocarbon group. R is a hydrocarbon group containing a branched structure and the branched chain is straight-chain C₆-C₁₀ alkyl group.

Further, R is a C₁₆-C₂₄ hydrocarbon group. R is an alkyl group containing a branched structure and the branched chain is a straight-chain C₆-C₁₀ alkyl group.

When R is an unsaturated hydrocarbon group, the unsaturated hydrocarbon group contains 1-5 alkenyl groups, or 1-5 alkynyl groups, or 1-5 the alkenyl groups and the alkynyl groups.

The branched fatty alcohol is one of the 2-hexyl-octanol, 1-heptyl-octanol, 2-hexyl-decanol, 1-butyl-dodecanol, 1-heptyl-nonanol, 1-octyl-nonanol, 2-octyl-decanol, 2-heptyl-undecanol, 1-nonyl-decanol, 2-octyl-dodecanol, 2-decyl-tetradecanol or 2-dodecyl-tetradecanol.

Preferably, the branched fatty alcohol is 2-hexyl-decanol, 2-heptyl-undecanol, 2-octyl-dodecanol or 2-decyl-tetradecanol.

The cabazitaxel and the branched fatty alcohol in the small molecule cabazitaxel prodrugs with branched fatty alcohol are linked by dibasic acid as a linking chain. The dibasic acid can be a monosulfuric dibasic acid, a monoselenicdibasic acid, or a dithiobasic acid. Specifically, the monosulfuric dibasic acid can be thiodiacetic acid, thiodipropionic acid, or thiodibutyric acid; the monoselenic dibasic acid is monoselenic diacetic acid, monoselenic dipropionic acid or monoselenic dibutyric acid; and the dithiobasic acid is 2,2′-dithiodiglycolic acid, 3,3′-dithiodipropionic acid or 4,4′-dithiodibutyric acid.

Specifically, the present invention provides cabazitaxel-2-hexyl-decanol prodrug, cabazitaxel-2-heptyl-undecanol prodrug, cabazitaxel-2-octyl-dodecanol prodrug, and cabazitaxel-2-decyl-tetradecanol prodrug. 2,2′-dithiodiglycolic acid is selected as the linking chain, and the corresponding prodrug is named as CTX-SS-HD, CTX-SS-HU, CTX-SS-OD, and CTX-SS-DT, respectively. Their structural formulas are shown as follows:

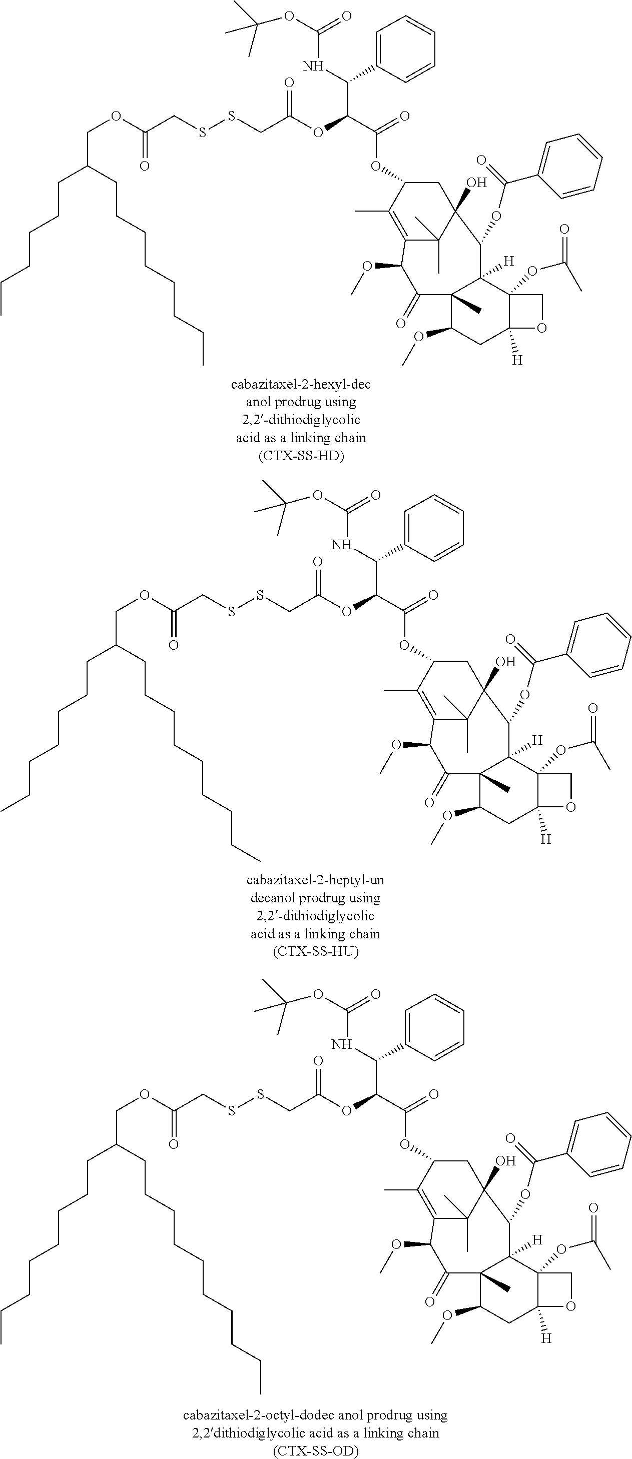

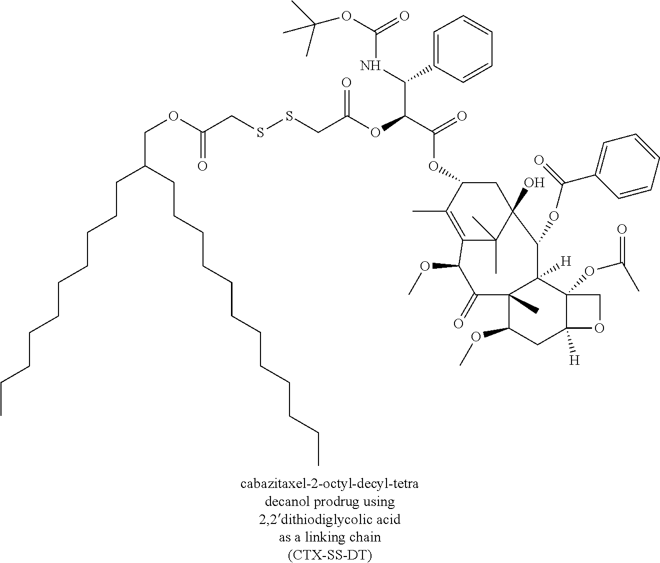

The present invention provides a small molecule cabazitaxel prodrug with straight chain fatty alcohol, that is cabazitaxel-arachidic alcohol prodrug. 2,2′-dithiodiglycolic acid is selected as the linking chain and the corresponding prodrug is named as CTX-SS-AA with the structural formula below:

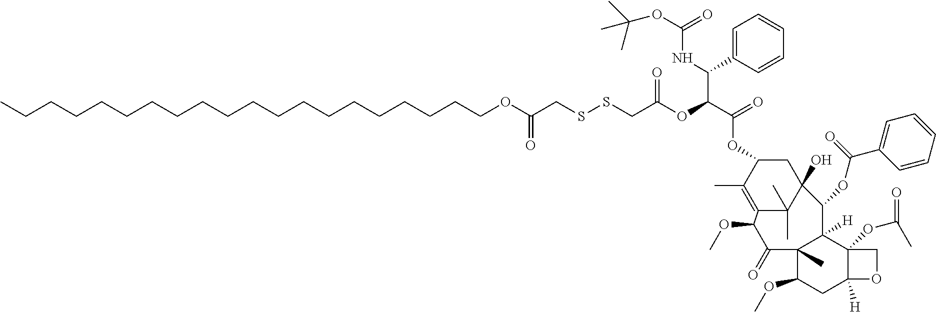

cabazitaxel-arachidic alcohol prodrug (CTX-SS-AA) uses 2,2′-dithiobisacetic acid as linking chain.

The present invention further provides a cabazitaxel-2-octyl-dodecanol prodrug using a 4,4′-dithiodibutyric acid as a linking chain. The corresponding prodrug is named as γ-CTX-SS-OD, with the structural formula below:

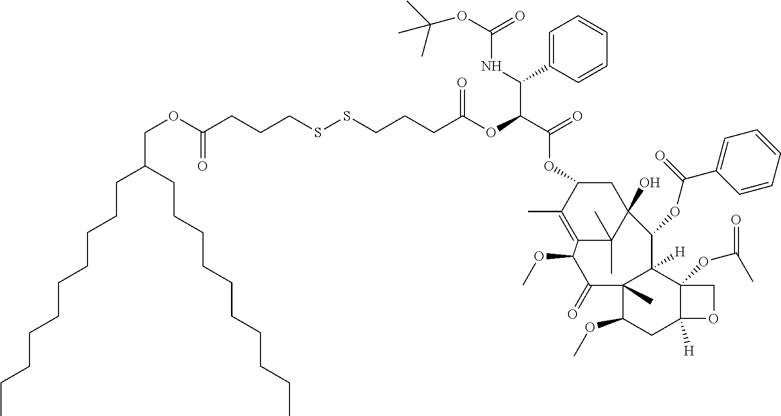

cabazitaxel-2-octyl-dodecanol prodrug uses 4,4′-dithiodibutyric acid as a linking chain (γ-CTX-SS-OD).

The present invention further provides cabazitaxel-2-octyl-dodecanol prodrugs using monosulfuric diacetic acid and monoselenic diacetic acid as linking chains, respectively. The corresponding prodrugs are named as CTX-S-OD and CTX-Se-OD, respectively, with the structural formulas below:

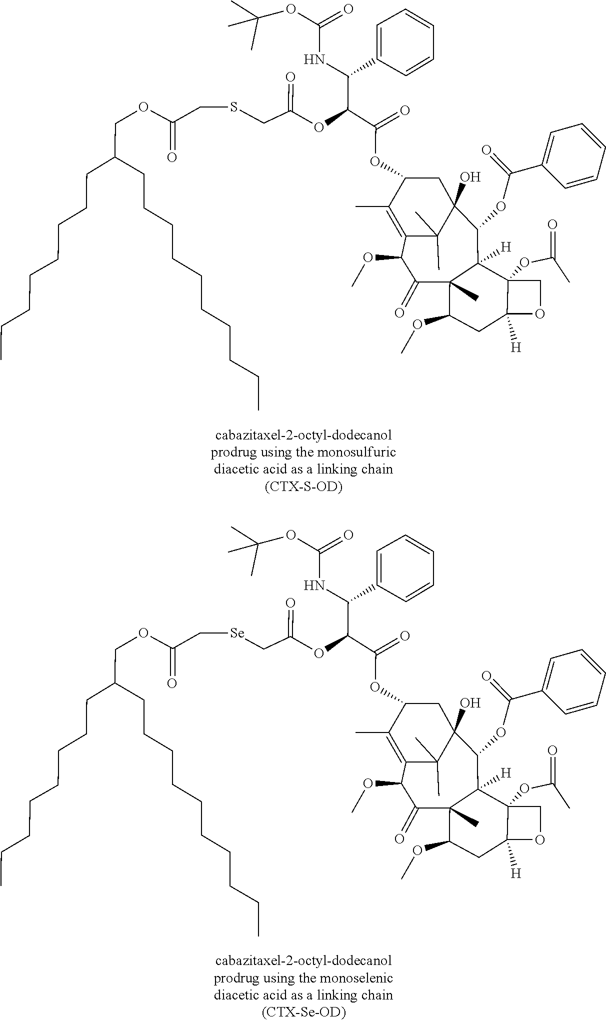

A synthetic method for the small molecule cabazitaxel prodrug with branched fatty alcohol includes the following steps:

Step 1: after dissolving dibasic acid to obtain dibasic anhydride, an esterification reaction is performed with branched fatty alcohol to obtain an intermediate product, namely a branched fatty alcohol-dibasic acid monolateral ester. The molar ratio of the branched fatty alcohol to the dibasic anhydride is (1-10): (5-15). The dibasic acid can be the monosulfuric dibasic acid, the monoselenic dibasic acid or the dithiobasic acid;

Step 2: enabling the branched fatty alcohol-dibasic acid monolateral ester and the cabazitaxel to be subjected to an ester-forming reaction to obtain an end product, namely the small molecule cabazitaxel prodrug with branched fatty alcohol. The molar ratio of the branched fatty alcohol-dibasic acid monolateral ester to the cabazitaxel is 1: (0.5-10) as shown in the following reaction equation:

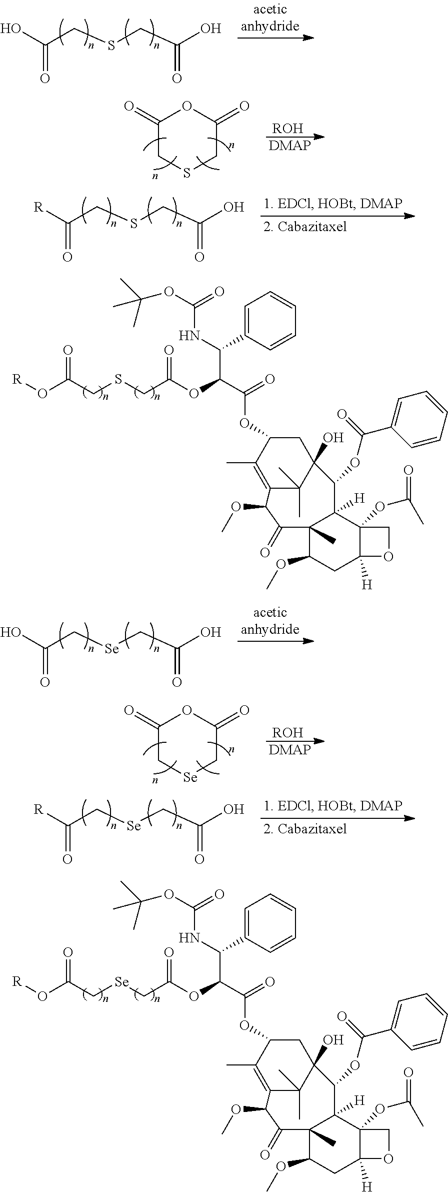

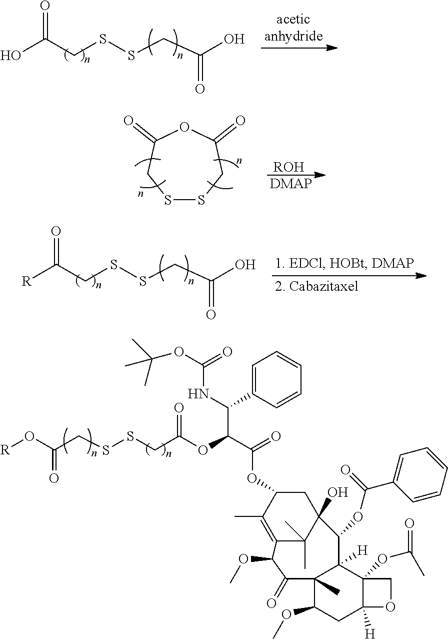

where n=1-3;
R can be either a saturated or unsaturated C₃-C₃₀ hydrocarbon group. R is a hydrocarbon group containing a branched structure and the branched chain is one or more of a C₁-C₁₈ alkyl group, a C₂-C₁₈ alkenyl group, or a C₂-C₁₈ alkynyl group.

The synthetic method for the small molecule cabazitaxel prodrug with branched fatty alcohols specifically includes the following steps:
(1) dissolve the dibasic acid in acetic anhydride and stir at room temperature for 2-4 hours to convert the dibasic acid into dibasic anhydride. After the reaction has completed, add toluene and remove toluene and the acetic anhydride by rotary evaporation under reduced pressure;
(2) dissolve branched fatty alcohol, 4-dimethylaminopyridine (DMAP), and the dibasic anhydride obtained in step (1) in dichloromethane. After performing stirring at room temperature for 12-18 hours, the intermediate product was separated and obtained by column chromatography, namely branched fatty alcohol-dibasic acid monolateral ester;
(3) dissolve n-(3-dimethylaminopropyl)-n′-ethylcarbodiimide hydrochloride (EDCI), 1-hydroxybenzotriazole (HOBt), the 4-dimethylaminopyridine (DMAP), and the intermediate product (the branched fatty alcohol-dibasic acid monolateral ester) in anhydrous dichloromethane and stir in an ice bath for 2-4 hours. Then, add cabazitaxel and stir at room temperature for another 24-48 hours. Finally, a reverse-phase preparative high performance liquid chromatography was applied to separate and purify the end product, namely the small molecule cabazitaxel prodrug with branched fatty alcohol.

The entire process of reaction for the synthetic method of the small molecule cabazitaxel prodrug with branched fatty alcohol is performed under nitrogen protection.

In the step (1), the mentioned dibasic acid can be the monosulfuric diacetic acid, the monosulfuric dipropionic acid, the monosulfuric dibutyric acid, the monoselenic diacetic acid, the monoselenic dipropionic acid, the monoselenic dibutyric acid, the 2,2′-dithiodiglycolic acid, the 3,3′-dithiodipropionic acid or the 4,4′-dithiodibutyric acid.

In the step (1), the proportion of the dibasic acid to the acetic anhydride is 1: (1-10), preferably 1: (1-2) in mmol: mL. In the step (2), the branched fatty alcohol can be either the C₃-C₃₀ saturated or unsaturated fatty alcohol. The branched chain is one or more of the C₁-C₁₈ alkyl group, the C₂-C₁₈ alkenyl group or the C₂-C₁₈ alkynyl group.

In the step (2), the molar ratio of the DMAP to the branched fatty alcohol to the dibasic anhydride is 1: (1-10):(5-15), preferably 1: (2-5):(10-15).

In the step (3), the molar ratio of the intermediate product namely the branched fatty alcohol-dibasic acid monolateral ester to the HOBt to the EDCI to the DMAP to the cabazitaxel is 1: (1-10):(2-6):(0.2-5):(0.5-10), preferably 1: (1-2): (2-4):(0.5-2):(0.8-2).

In the step (3), the purity of the prepared small molecule cabazitaxel prodrug with branched fatty alcohol is more than 99%.

The present invention also provides a synthetic method for the monoselenic diacetic acid, including the following steps:

Add selenium powder and a small amount of water to an eggplant-shaped bottle in an ice water bath, slowly dropwise add a sodium borohydride aqueous solution, and stir until the solution is clear and transparent. Raise the temperature to 100-110° C. and stir for another 30-45 minutes. After that, temperature is reduced to room temperature and a bromoacetic acid aqueous solution is dropwise added. After reaction for 10-12 hours, the reaction solution is filtered and extracted by adding ethyl acetate until the aqueous layer is colorless. Remove the ethyl acetate by rotary evaporation under reduced pressure to obtain the final product. The entire process of the reaction is performed under nitrogen protection.

The present invention further provides a self-assembled nanoparticle of the small molecule cabazitaxel prodrug with branched fatty alcohol. The self-assembled nanoparticle of the prodrug is a non-PEGylated prodrug-based self-assembled nanoparticle, a PEGylated/active targeting prodrug-based self-assembled nanoparticle, or a hydrophobic fluorescent substances/drugs and prodrug co-assembled nanoparticle.

The preparation method of the small molecule cabazitaxel-branched fatty alcohol prodrug-based self-assembled nanoparticles includes the following steps:

When the prodrug-based self-assembled nanoparticle is the non-PEGylated one, the preparation method is as follows. Dissolve a certain quantity of the prodrug into an appropriate quantity of an organic solvent and dropwise add the solution into water under stirring. The prodrug spontaneously self-assembles into uniform nanoparticle. After removing the organic solvent by vacuum rotary evaporation, a nanocolloidal solution is obtained without any organic solvents, namely the non-PEGylated small molecule cabazitaxel fatty alcohol prodrug-based self-assembled nanoparticles.

A preparation method for the PEGylated/active targeting small molecule cabazitaxel-branched fatty alcohol prodrug-based self-assembled nanoparticles is as follows. Dissolve a certain quantity of the PEG modifier/active targeting modifier and the prodrug in an appropriate quantity of the organic solvent. Then, the solvents are slowly dropwise added into water under stirring. The prodrug spontaneously self-assembles into uniform nanoparticles. After removing the organic solvent by vacuum rotary evaporation, the nanocolloidal solution without any organic solvents is obtained, namely the PEGylated/active targeting small molecule cabazitaxel fatty alcohol prodrug-based self-assembled nanoparticles. Thereinto, the mass ratio of small molecule cabazitaxel prodrug with branched fatty alcohol to a PEG modifier/active targeting modifier is 1: (0.1-1). The PEG modifier is an amphiphilic polymer or a targeting group, such as DSPE-PEG, TPGS, PLGA-PEG, PE-PEG, or DSPE-PEG-FA. The active targeting modifier is substances capable of targeting specific tissue, such as antibody, sugar residue, hormone, receptor or ligand.

When the self-assembled nanoparticle is the hydrophobic fluorescent substances/drugs and small molecule cabazitaxel-branched fatty alcohol prodrug co-assembled nanoparticle, the preparation method is as follows. Dissolve a certain quantity of the PEG modifier, the hydrophobic fluorescent substances/drugs, and the small molecule cabazitaxel-branched fatty alcohol prodrug into an appropriate quantity of the organic solvent. Then, the solvents are dropwise added into water under stirring. The prodrug spontaneously self-assembles into uniform nanoparticle. After removing the organic solvent by vacuum rotary evaporation, the nanocolloidal solution without any organic solvents is obtained, namely the hydrophobic fluorescent substances/drugs and small molecule cabazitaxel-branched fatty alcohol prodrug co-assembled nanoparticle. Thereinto, the mass ratio of the small molecule cabazitaxel prodrug with branched fatty alcohol to the PEG modifier, and the hydrophobic fluorescent substances/drugs is 1: (0.1-1):(0.1-1).

The present invention further provides application of the small molecule cabazitaxel prodrugs with branched fatty alcohol or their self-assembled nanoparticles in the field of anti-tumor drugs.

The present invention further provides application of the small molecule cabazitaxel prodrugs with branched fatty alcohol or their self-assembled nanoparticles in the field of injection administration, oral administration, or topical administration system.

The present invention further provides application of the small molecule cabazitaxel prodrugs with branched fatty alcohol or their self-assembled nanoparticles in the field of drug delivery system with improving efficacy and reducing toxicity.

A lyophilized powder of the small molecule cabazitaxel-branched fatty alcohol prodrug self-assembled nanoparticles includes the small molecule cabazitaxel-branched fatty alcohol prodrug self-assembled nanoparticles and lyophilized protective agents. The concentration of prodrug-based self-assembled nanoparticles solution ranges from 0.1 mg/mL to 20 mg/mL. The lyophilized protective agent is one or more of monose including glucose and galactose, disaccharide including trehalose and sucrose, polyalcohol including mannitol, sorbitol and xylitol, and polymers including polyethylene glycol, hydroxyethyl starch, or dextran. The quantity of the lyophilized protective agent ranges from 1%-20% (W/V) (namely, the mass concentration of the lyophilized protective agent ranges from 10 g/L to 200 g/L), preferably 5%-10% (W/V).

A preparation method for the lyophilized powder of the small molecule cabazitaxel-branched fatty alcohol prodrug-based self-assembled nanoparticles includes the following steps:

Place the small molecule cabazitaxel-branched fatty alcohol prodrug-based self-assembled nanoparticles in a vial and add the lyophilized protective agents. After the lyophilized protective agent is completely dissolved, place the vial in the −80° C. environment to be pre-frozen for 8-12 hours. Then, place the pre-frozen solutions in a lyophilizer to be freeze-dried. After 24-72 hours, white cakes are obtained, namely the lyophilized powder of the small molecule cabazitaxel-branched fatty alcohol prodrug-based self-assembled nanoparticles.

The present invention has the beneficial effects:

(1) The present invention designs and synthesizes the small molecule cabazitaxel-fatty alcohol prodrugs containing different fatty alcohol side chains and different linking chains. The synthetic method is simple and easy to implement. The small molecule cabazitaxel fatty alcohol prodrug-based self-assembled nanoparticles are also prepared with the characterizations of small particle size, uniform size distribution and the preparation method is simple and easy to implement. (2) The effects of branched fatty alcohol side chains of four lengths, the structure of the fatty alcohol side chains (branched or straight-chain), and the four linking chains on the pharmaceutical properties, in vivo fate, anti-tumor activity, etc. of the prodrug-based self-assembled nanoparticles are investigated. Results showed that the small molecule cabazitaxel fatty alcohol prodrug-based self-assembled nanoparticles can effectively improve the anti-tumor efficacy and reduce the toxicity of cabazitaxel. Different side chains and linking chains will significantly affect the pharmaceutical properties, in vivo fate, and anti-tumor activity of the cabazitaxel prodrug-based self-assembled nanoparticles. The prodrug-based self-assembled nanoparticles use the 2-octyl-dodecanol as the side chain possess superior safety. The small molecule cabazitaxel-branched fatty alcohol prodrug-based self-assembled nanoparticles display better anti-tumor effects and safety than small molecule cabazitaxel-straight fatty alcohol prodrug-based self-assembled nanoparticles. The small molecule cabazitaxel prodrug-based self-assembled nanoparticles using disulfide bond as the linking chain are more advantageous in terms of anti-tumor effects. The small molecule cabazitaxel prodrug-based self-assembled nanoparticles using 4,4′-dithiodibutyric acid as the linking chain and 2-octyl-dodecanol as side chain exhibit the most potent activity in anti-tumor activity with superior safety and the highest tolerated dose. The present invention provides new strategies and options for the development of highly effective-low toxicity chemotherapy preparations.

### BRIEF DESCRIPTION OF DRAWINGS

FIG. 1 shows a graph of tumor volume changes in vivo anti-tumor assay of the PEG-modified small molecule prodrug-based self-assembled nanoparticles in Example 13 of the present invention.

n.s.: P≥0.05 *: P<0.05 **: P<0.01 ***: P<0.001 ****: P<0.0001 (Student's t test (two-tailed))

FIG. 2 shows a graph of mice weight changes in vivo anti-tumor assay of the PEG-modified small molecule prodrug-based self-assembled nanoparticles in Example 13 of the present invention.

n.s.: P≥0.05 *: P<0.05 **: P<0.01 ***: P<0.001 ****: P<0.0001 (Student's t test (two-tailed))

FIG. 3 shows a graph of blood routine indexes in vivo anti-tumor assay of the PEG-modified small molecule prodrug-based self-assembled nanoparticles in Example 13 of the present invention.

FIG. 4 shows a graph of tumor volume changes in vivo anti-tumor assay of the PEG-modified small molecule prodrug-based self-assembled nanoparticles in Example 14 of the present invention.

n.s.: P≥0.05 *: P<0.05 **: P<0.01 ***: P<0.001 ****: P<0.0001 (Student's t test (two-tailed))

FIG. 5 shows a graph of mice weight changes in vivo anti-tumor assay of the PEG-modified small molecule prodrug-based self-assembled nanoparticles in Example 14 of the present invention.

n.s.: P≥0.05 *: P<0.05 **: P<0.01 ***: P<0.001 ****: P<0.0001 (Student's t test (two-tailed))

FIG. 6 shows a graph of tumor burden in vivo anti-tumor assay of the PEG-modified small molecule prodrug-based self-assembled nanoparticles in Example 14 of the present invention.

n.s.: P≥0.05 *: P<0.05 **: P<0.01 ***: P<0.001 ****: P<0.0001 (Student's t test (two-tailed))

FIG. 7 shows a graph of blood routine indexes in vivo anti-tumor assay of the PEG-modified small molecule prodrug-based self-assembled nanoparticles in Example 14 of the present invention.

FIG. 8 shows a graph of biochemical routine indexes in vivo anti-tumor assay of the PEG-modified small molecule prodrug-based self-assembled nanoparticles in Example 14 of the present invention.

FIG. 9 shows a graph of tumor volume changes in vivo anti-tumor assay of the PEG-modified small molecule prodrug-based self-assembled nanoparticles in Example 15 of the present invention.

n.s.: P≥0.05 *: P<0.05 **: P<0.01 **** P<0.001 ****: P<0.0001 (Student's t test (two-tailed))

FIG. 10 shows a graph of mice weight changes in vivo anti-tumor assay of the PEG-modified small molecule prodrug-based self-assembled nanoparticles in Example 15 of the present invention.

n.s.: P≥0.05 *: P<0.05 **: P<0.01 *: P<0.001 ****: P<0.0001 (Student's t test (two-tailed))

FIG. 11 shows a graph of tumor burden in vivo anti-tumor assay of the PEG-modified small molecule prodrug-based self-assembled nanoparticles in Example 15 of the present invention.

n.s.: P≥0.05 *: P<0.05 **: P<0.01 ***: P<0.001 ****: P<0.0001 (Student's t test (two-tailed))

FIG. 12 shows a graph of tumor volume changes in vivo anti-tumor assay of the PEG-modified small molecule prodrug-based self-assembled nanoparticles in Example 16 of the present invention.

n.s.: P≥0.05 *: P<0.05 **: P<0.01 ***: P<0.001 ****: P<0.0001 (Student's t test (two-tailed))

FIG. 13 shows a graph of mice weight changes in vivo anti-tumor assay of the PEG-modified small molecule prodrug-based self-assembled nanoparticles in Example 16 of the present invention.

n.s.: P≥0.05 *: P<0.05 **: P<0.01 *: P<0.001 ****: P<0.0001 (Student's t test (two-tailed))

FIG. 14 shows a graph of tumor burden in vivo anti-tumor assay of the PEG-modified small molecule prodrug-based self-assembled nanoparticles in Example 16 of the present invention.

n.s.: P≥0.05 *: P<0.05 **: P<0.01 ***: P<0.001 ****: P<0.0001 (Student's t test (two-tailed))

FIG. 15 shows a graph of tumor volume changes in vivo anti-tumor assay of the PEG-modified small molecule prodrug-based self-assembled nanoparticles in Example 17 of the present invention.

n.s.: P≥0.05 *: P<0.05 **: P<0.01 ***: P<0.001 ****: P<0.0001 (Student's t test (two-tailed))

FIG. 16 shows a graph of mice weight changes in vivo anti-tumor assay of the PEG-modified small molecule prodrug-based self-assembled nanoparticles in Example 17 of the present invention.

n.s.: p≥0.05 *: p<0.05 **: p<0.01 ***: p<0.001 ****: p<0.0001 (Student's t test (two-tailed)).

### DETAILED DESCRIPTION OF THE PREFERRED EMBODIMENT

The present invention is described in further detail below in connection with examples.

### Example 1: Synthesis of Cabazitaxel-2-Hexyl-Decanol Prodrug CTX-SS-HD Using 2,2′-Dithiobisacetic Acid as Linking Chain

An appropriate quantity of 2,2-dithiodiacetic acid was dissolved in a 25 mL round-bottomed flask containing acetic anhydride, and reacted for 2 h with magnetic stirring. The reaction solution is transferred to a 100 mL round-bottomed flask containing toluene of three times the quantity. The toluene and acetic anhydride in the reaction solution were removed by vacuum rotary evaporation to obtain dithioacetic anhydride. After mixing dithiodiacetic anhydride dissolved in dichloromethane with 2-hexyl-decanol, a dichloromethane solution of 4-dimethylaminopyridine (DMAP) was slowly added dropwise to the reaction solution. The reaction was carried out under magnetic stirring for 12 h to obtain the intermediate product 2-hexyl-decanol-dithiodiacetic acid monoester. Then, chromatography column method was employed to separate and purify of 2-hexyl-decanol-dithiodiacetic acid monoester, with cyclohexane-acetone as the mobile phase. The purified previous product, n-(3-dimethylaminopropyl)-n′-ethylcarbodiimide hydrochloride (EDCI), 1-hydroxybenzotriazole (HOBt) and 4-dimethylaminopyridine (DMAP) were dissolved in dichloromethane, activated in an ice bath for 2 h, and then added CTX with stirring at 25° C. for another 48 h. After reaction was completed, the products were separated and purified by the preparative liquid obtaining cabazitaxel-2-hexyl-decanol prodrug using the 2,2′-dithiobisacetic acid as a linking chain. In the reaction, the proportion of the 2,2′-dithiobisacetic acid to the acetic anhydride is 1:1, in mmol: ml; a molar ratio of the DMAP to the 2-hexyl-decanol to the dithiobisaceticacid anhydride is 0.4:2:1, and the proportion of the 2-hexyl-decanol-dithiobisacetic acid monolateral ester to HOBt to EDCI to DMAP to cabazitaxel is 1:1:2:0.4:0.8.

Mass spectrometry and ¹H-NMR were used to corroborate the structure of the products. The results of the spectral analysis were as follows:

¹H-NMR (600 MHZ, DMSO-d₆) δ7.966 (2H, d), 7.840 (1H, m,), 7.650-7.677 (2H, m,), 7.348 (2H, m), 7.174 (3H, m), 5.812 (1H, s, 13-H), 5.070-5.131 (2H, d, 2-H, 3′-H), 4.954 (1H, d, 2′-H), 4.931 (3H, m, 3-H, —CH═CH—), 4.686 (1H, d, 4-H), 4.459 (1H, m, 5-H), 4.013 (1H, d, 20α-H), 3.996 (4H, m, —OCH₂CH₂O—), 3.857 (1H, d, 7-H), 3.590 (1H, d, 8-H), 3.291 (4H, m, —CH₂-SS-CH₂—), 3.207 (4H, m, 6α-H, s, 4-COCHCH₂), 2.650 (1H, m, 14α-H), 2.236-2.495 (2H, m, 14β-H, 15α-H), 1.791 (7H, t, 6β-H, —CH₂CH=CHCH₂—), 1.504 (5H,s, 19-H, 10-COCH₃), 1.376 (6H,m, —CH₂CH₂CH₂CO—), 1.245 (31H, t, 17-H), 0.978 (7H, s, 16-H), 0.852 (6H, t, —CH₃).

MS (ESI) m/z for C₆₅H₉₃NO₁₇S₂Na [M+Na]⁺: 1246.

### Example 2: Synthesis of Cabazitaxel-2-Heptyl-Undecanol Prodrug Using 2,2′-Dithiobisacetic Acid as Linking Chain

An appropriate quantity of 2,2-dithiodiacetic acid was dissolved in a 25 mL round-bottomed flask containing acetic anhydride, and reacted for 2 h with magnetic stirring. The reaction solution is transferred to a 100 mL round-bottomed flask containing toluene of three times the quantity. The toluene and acetic anhydride in the reaction solution were removed by vacuum rotary evaporation to obtain dithioacetic anhydride. After mixing dithiodiacetic anhydride dissolved in dichloromethane with 2-heptyl-undecanol, a dichloromethane solution of 4-dimethylaminopyridine (DMAP) was slowly added dropwise to the reaction solution. The reaction was carried out under magnetic stirring for 12 hours to obtain the intermediate product 2-heptyl-undecanol-dithiodiacetic acid monoester. Then, chromatography column method was employed to separate and purify of 2-heptyl-undecanol-dithiodiacetic acid monoester, with cyclohexane-acetone as the mobile phase. The purified previous product, n-(3-dimethylaminopropyl)-n′-ethylcarbodiimide hydrochloride (EDCI), 1-hydroxybenzotriazole (HOBt) and 4-dimethylaminopyridine (DMAP) were dissolved in dichloromethane, activated in an ice bath for 2 h, and then added CTX with stirring at 25° C. for another 48 h. After reaction was completed, the products were separated and purified by the preparative liquid phase, obtaining cabazitaxel-2-heptyl-undecanol prodrug linked by the 2,2′-dithiobisacetic acid. In the reaction, the proportion of the 2,2′-dithiobisacetic acid to the acetic anhydride is 1:1, in mmol: ml; the molar ratio of the DMAP to the 2-heptyl-undecanol to the dithiobisacetic acid anhydride is 0.4:2:1, and the proportion of the 2-heptyl-undecanol-dithiobisacetic acid monolateral ester to HOBt to EDCI to DMAP to cabazitaxel is 1 to 1:2:0.4:0.8.

Mass spectrometry and ¹H-NMR were used to corroborate the structure of the products. The results of the spectral analysis were as follows:

¹H-NMR (600 MHZ, DMSO-d₆) δ7.967 (2H, d), 7.817 (1H, m), 7.737 (2H, m), 7.658 (2H, m), 7.175 (3H, m), 5.814 (1H, s, 13-H), 5.131-5.358 (2H, d, 2-H, 3′-H), 5.071 (1H, d, 2′-H), 4.932 (3H, m, 3-H, —CH=CH—), 4.687 (1H, d, 4-H), 4.460 (1H, m, 5-H), 4.015 (1H, d, 20α-H), 3.976 (4H, m, —OCH₂CH₂O—), 3.857 (1H, d, 7-H), 3.574 (1H, d, 8-H), 3.293 (4H, m, —CH₂-SS-CH₂—), 3.207 (4H, m, 6α-H, s, 4-COCHCH₂), 2.651 (1H, d, 18-H), 2.237-2.500 (2H, m, 14β-H, 15α-H), 1.792 (7H, t, 6β-H, —CH₂CH=CHCH₂—), 1.505 (5H, s, 19-H, 10-COCH₃), 1.377 (6H, m, —CH₂CH₂CH₂CO—), 1.243 (35H, t, 17-H), 0.979 (7H, s, 16-H), 0.853 (6H, t, —CH₃).

MS (ESI) m/z for C₆₇H₉₇NO₁₇S₂Na [M+Na]⁺: 1276.

### Example 3: Synthesis of Cabazitaxel-2-Octyl-Dodecanol Prodrug CTX-SS-HD Using 2,2′-Dithiobisacetic Acid as Linking Chain

An appropriate quantity of 2,2-dithiodiacetic acid was dissolved in a 25 mL round-bottomed flask containing acetic anhydride, and reacted for 2 h with magnetic stirring. The reaction solution was transferred to a 100 mL round-bottomed flask containing toluene of three times the quantity. The toluene and acetic anhydride in the reaction solution were removed by vacuum rotary evaporation to obtain dithioacetic anhydride. After mixing dithiodiacetic anhydride dissolved in dichloromethane with 2-octyldodecanol, a dichloromethane solution of 4-dimethylaminopyridine (DMAP) was slowly added dropwise to the reaction solution. The reaction was carried out under magnetic stirring for 12 hours to obtain the intermediate product 2-octyldodecyl-dithiodiacetic acid monoester. Then, chromatography column method was employed to separate and purify of 2-octyldodecyl-dithiodiacetic acid monoester, with cyclohexane-acetone as the mobile phase. The purified previous product, n-(3-dimethylaminopropyl)-n′-ethylcarbodiimide hydrochloride (EDCI), 1-hydroxybenzotriazole (HOBt) and 4-dimethylaminopyridine (DMAP) were dissolved in dichloromethane, activated in an ice bath for 2 h, and then added CTX with stirring at 25° C. for another 48 h. After reaction was completed, the products were separated and purified by the preparative liquid phase, obtaining cabazitaxel-2-octyl-dodecanol prodrug using a 2,2′-dithiobisacetic acid as a linking chain. In the reaction, the proportion of the 2,2′-dithiobisacetic acid to the acetic anhydride is 1:1, in mmol: ml; the molar ratio of the DMAP to the 2-octyl-dodecanol to the dithiobisacetic acid anhydride is 0.4:2:1, and the proportion of the 2-octyl-dodecanol-dithiobisacetic acid monolateral ester to HOBt to EDCI to DMAP to cabazitaxel is 1:1:2:0.4:0.8.

Mass spectrometry and ¹H-NMR were used to corroborate the structure of the products. The results of the spectral analysis were as follows: ¹H-NMR (600 MHZ, DMSO-d₆) δ7.967 (2H, t), 7.662 (3H, d), 7.378-7.423 (3H, m), 7.153-7.189 (2H, m), 5.807 (1H, d, —NH), 5.355 (1H, d, 3′-H), 5.124 (1H, d, 2-H), 5.065 (4H, m, 2′-H, —CH-CH—, 5-H), 4.924-4.954 (1H, t, 7-H), 4.685 (1H, d, 20α-H), 4.477 (1H, d, 20β-H), 3.993-4.012 (2H, d, 15α-H, 15β-H), 3.861 (3H, d, 3-H), 3.736 (4H, m, 6α-H, 4-COCH₃), 2.647 (1H, m, 13-H), 2.501 (3H, t, 14α-H, —CH₂CO—), 1.790 (6H, s, —CH₂CH-CHCH₂—), 1.618 (4H, s, 18-H), 1.503 (t, 5H, 6β-H), 1.377 (10H, s, 16-H, 19-H, —CH₂CH₂CO—), 1.240 (24H, t, 17-H), 0.975 (3H, t, —CH₃), 0.850 (5H, t, —CH₂CH₃).

MS (ESI) m/z for C₆₉H₁₀₁NO₁₇S₂Na [M+Na]⁺: 1303.

### Example 4: Synthesis of Cabazitaxel-2-Decyl-Tetradecanol Prodrug Using 2,2′-Dithiobisacetic Acid as Linking Chain

An appropriate quantity of 2,2-dithiodiacetic acid was dissolved in a 25 mL round-bottomed flask containing acetic anhydride, and reacted for 2 h with magnetic stirring. The reaction solution is transferred to a 100 mL round-bottomed flask containing toluene of three times the quantity. The toluene and acetic anhydride in the reaction solution were removed by vacuum rotary evaporation to obtain dithioacetic anhydride. After mixing dithiodiacetic anhydride dissolved in dichloromethane with 2-decyl-tetradecanol, a dichloromethane solution of 4-dimethylaminopyridine (DMAP) was slowly added dropwise to the reaction solution. The reaction was carried out under magnetic stirring for 12 hours to obtain the intermediate product 2-decyl-tetradecanol-dithiodiacetic acid monoester. Then, chromatography column method was employed to separate and purify of 2-decyl-tetradecanol-dithiodiacetic acid monoester, with cyclohexane-acetone as the mobile phase. The purified previous product, n-(3-dimethylaminopropyl)-n′-ethylcarbodiimide hydrochloride (EDCI), 1-hydroxybenzotriazole (HOBt) and 4-dimethylaminopyridine (DMAP) were dissolved in dichloromethane, activated in an ice bath for 2 h, and then added CTX with stirring at 25° C. for another 48 h. After reaction was completed, the products were separated and purified by the preparative liquid phase, obtaining cabazitaxel-2-decyl-tetradecanol prodrug using the 2,2′-dithiobisacetic acid as a linking chain. In the reaction, the proportion of the 2,2′-dithiobisacetic acid to the acetic anhydride is 1:1, in mmol: ml; and the molar ratio of the DMAP to the 2-decyl-tetradecanol to the dithiobisacetic acid anhydride is 0.4:2:1, and the proportion of the 2-decyl-tetradecanol-dithiobisacetic acid monolateral ester to HOBt to EDCI to DMAP to cabazitaxel is 1:1:2:0.4:0.8.

Mass spectrometry and ¹H-NMR were used to corroborate the structure of the products. The results of the spectral analysis were as follows:

¹H-NMR (600 MHZ, DMSO-d₆) δ7.967 (2H, d), 7.817 (1H, m), 7.737 (2H, m), 7.658 (2H, m), 7.175 (3H, m), 5.814 (1H, s, 13-H), 5.131-5.358 (2H, d, 2-H, 3′-H), 5.071 (1H, d, 2′-H), 4.932 (3H, m, 3-H, —CH=CH—), 4.687 (1H, d, 4-H), 4.460 (1H, m, 5-H), 4.015 (1H, d, 20α-H), 3.976 (4H, m, —OCH₂CH₂O—), 3.857 (1H, d, 7-H), 3.574 (1H, d, 8-H), 3.293 (4H, m, —CH₂-SS-CH₂—), 3.207 (4H, m, 6α-H, s, 4-COCHCH₂), 2.651 (1H, d, 18-H), 2.237-2.500 (2H, m, 14β-H, 15α-H), 1.792 (7H, t, 6β-H, —CH₂CH═CHCH₂—), 1.505 (5H, s, 19-H, 10-COCH₃), 1.377 (6H, m, —CH₂CH₂CH₂CO—), 1.243 (35H, t, 17-H), 0.979 (7H, s, 16-H), 0.853 (6H, t, —CH₃).

MS (ESI) m/z for C₇₃H₁₀₉NO₁₇S₂Na [M+Na]⁺: 1359.

### Example 5: Synthesis of Cabazitaxel-Arachidic Alcohol Prodrug Using 2,2′-Dithiobisacetic Acid as Linking Chain

An appropriate quantity of 2,2-dithiodiacetic acid was dissolved in a 25 mL round-bottomed flask containing acetic anhydride, and reacted for 2 h with magnetic stirring. The reaction solution is transferred to a 100 mL round-bottomed flask containing toluene of three times the quantity. The toluene and acetic anhydride in the reaction solution were removed by vacuum rotary evaporation to obtain dithioacetic anhydride. After mixing dithiodiacetic anhydride dissolved in dichloromethane with arachidic alcohol, a dichloromethane solution of 4-dimethylaminopyridine (DMAP) was slowly added dropwise to the reaction solution. The reaction was carried out under magnetic stirring for 12 hours to obtain the intermediate product arachidic alcohol-dithiodiacetic acid monoester. Then, chromatography column method was employed to separate and purify of arachidic alcohol-dithiodiacetic acid monoester, with cyclohexane-acetone as the mobile phase. The purified previous product, n-(3-dimethylaminopropyl)-n′-ethylcarbodiimide hydrochloride (EDCI), 1-hydroxybenzotriazole (HOBt) and 4-dimethylaminopyridine (DMAP) were dissolved in dichloromethane, activated in an ice bath for 2 h, and then added CTX with stirring at 25° C. for another 48 h. After reaction was completed, the products were separated and purified by the preparative liquid phase, obtaining cabazitaxel-arachidic alcohol prodrug using the 2,2′-dithiobisacetic acid as a linking chain. In the reaction, the proportion of the 2,2′-dithiobisacetic acid to the acetic anhydride is 1:1, in mmol: ml; and the molar ratio of the DMAP to the arachidic alcohol to the dithiobisacetic acid anhydride is 0.4:2:1, arachidic alcohol-dithiobisacetic acid monolateral ester to HOBt to EDCI to DMAP to cabazitaxel is 1:1:2:0.4:0.8.

Mass spectrometry and ¹H-NMR were used to corroborate the structure of the products. The results of the spectral analysis were as follows: ¹H-NMR (600 MHZ, DMSO-d₆) δ7.967 (2H, t), 7.662 (3H, d), 7.378-7.423 (3H, m), 7.153-7.189 (2H, m), 5.807 (1H, d, —NH), 5.355 (1H, d, 3′-H), 5.124 (1H, d, 2-H), 5.065 (4H, m, 2′-H, —CH=CH—, 5-H), 4.924-4.954 (1H, t, 7-H), 4.685 (1H, d, 20α-H), 4.477 (1H, d, 20β-H), 3.993-4.012 (2H, d, 15α-H, 15β-H), 3.861 (3H, d, 3-H), 3.736 (4H, m, 6α-H, 4-COCH₃), 2.647 (1H, m, 13-H), 2.501 (3H, t, 14α-H, —CH₂CO—), 1.790 (6H, s, —CH₂CH=CHCH₂—), 1.618 (4H, s, 18-H), 1.503 (t, 5H, 6β-H), 1.377 (10H, s, 16-H, 19-H, —CH₂CH₂CO—), 1.240 (24H, t, 17-H), 0.975 (3H, t, —CH₃), 0.850 (5H, t, —CH₂CH₃).

MS (ESI) m/z for C₆₉H₁₀₁NO₁₇S₂Na [M+Na]⁺: 1280.

### Example 6: Synthesis of Cabazitaxel-2-Octyl-Dodecanol Prodrug Using 4,4′-Dithiodibutyric Acid as Linking Chain

An appropriate quantity of 4,4′-dithiodibutyric acid was dissolved in a 25 mL round-bottomed flask containing acetic anhydride, and reacted for 2 h with magnetic stirring. The reaction solution is transferred to a 100 mL round-bottomed flask containing toluene of three times the quantity. The toluene and acetic anhydride in the reaction solution were removed by vacuum rotary evaporation to obtain dithiodibutyrate anhydride. After mixing dithiodibutyrate anhydride dissolved in dichloromethane with 2-octyldodecanol, a dichloromethane solution of 4-dimethylaminopyridine (DMAP) was slowly added dropwise to the reaction solution. The reaction was carried out under magnetic stirring for 12 hours to obtain the intermediate product 2-octyldodecyl-dithiodibutyric acid monoester. Then, chromatography column method was employed to separate and purify of 2-octyldodecyl-dithiodibutyric acid monoester, with cyclohexane-acetone as the mobile phase. The purified previous product, n-(3-dimethylaminopropyl)-n′-ethylcarbodiimide hydrochloride (EDCI), 1-hydroxybenzotriazole (HOBt) and 4-dimethylaminopyridine (DMAP) were dissolved in dichloromethane, activated in an ice bath for 2 h, and then added CTX with stirring at 25° C. for another 48 h. After reaction was completed, the products were separated and purified by the preparative liquid phase, obtaining cabazitaxel-2-octyl-dodecanol prodrug using a 4,4′-dithiodibutyric acid as a linking chain. In the reaction, the proportion of the 4,4′-dithiodibutyric acid to the acetic anhydride is 1:1, in mmol: ml; and the molar ratio of the DMAP to the 2-octyl-dodecanolto the 4,4′-dithiodibutyric acid is 0.4:2:1, and the proportion of the 2-octyl-dodecanol-dithiobisacetic acid monolateral ester to HOBt to EDCI to DMAP to cabazitaxel is 1 to 1:2:0.4:0.8.

Mass spectrometry and ¹H-NMR were used to corroborate the structure of the products. The results of the spectral analysis were as follows: ¹H-NMR (600 MHZ, DMSO-d₆) δ7.965 (2H, t), 7.691 (3H, d), 7.349-7.425 (3H, m), 7.152-7.189 (2H, m), 5.753 (1H, d, —NH), 5.352 (1H, d, 3′-H), 5.120 (1H, d, 2-H), 5.026-5.069 (4H, m, 2′-H, —CH-CH—, 5-H), 4.927-4.951 (1H, t, 7-H), 4.682 (1H, d, 20α-H), 4.474 (1H, d, 20β-H), 3.986-4.011 (2H, d, 15α-H, 15-H), 3.583 (3H, d, 3-H), 3.495 (4H, m, 6α-H, 4-COCH₃), 2.670 (1H, m, 13-H), 2.501 (3H, t, 14α-H, —CH₂CO—), 1.780 (6H, s, —CH₂CH=CHCH₂—), 1.609 (4H, s, 18-H), 1.501 (t, 5H, 6β-H), 1.370 (10H, s, 16-H, 19-H, —CH₂CH₂CO—), 1.210 (24H, t, 17-H), 0.957 (3H, t, —CH₃), 0.831 (5H, t, —CH₂CH₃).

MS (ESI) m/z for C₇₃H₁₀₁NO₁₇S₂Na [M+Na]⁺: 1351.

### Example 7: Synthesis of Cabazitaxel-2-Octyl-Dodecanol Prodrug Using Thioether Bond as Linking Chain

An appropriate quantity of thiodiglycolic acid was dissolved in a 25 mL round-bottomed flask containing acetic anhydride, and reacted for 2 h with magnetic stirring. The reaction solution is transferred to a 100 mL round-bottomed flask containing toluene of three times the quantity. The toluene and acetic anhydride in the reaction solution were removed by vacuum rotary evaporation to obtain thioacetic anhydride. After mixing thioacetic anhydride dissolved in dichloromethane with 2-octyldodecanol, a dichloromethane solution of 4-dimethylaminopyridine (DMAP) was slowly added dropwise to the reaction solution. The reaction was carried out under magnetic stirring for 12 hours to obtain the intermediate product 2-octyldodecyl-thiodiglycolic acid monoester. Then, chromatography column method was employed to separate and purify of 2-octyldodecyl-thiodiglycolic acid monoester, with cyclohexane-acetone as the mobile phase. The purified previous product, n-(3-dimethylaminopropyl)-n′-ethylcarbodiimide hydrochloride (EDCI), 1-hydroxybenzotriazole (HOBt) and 4-dimethylaminopyridine (DMAP) were dissolved in dichloromethane, activated in an ice bath for 2 h, and then added CTX with stirring at 25° C. for another 48 h. After reaction was completed, the products were separated and purified by the preparative liquid phase, obtaining cabazitaxel-2-octyl-dodecanol prodrug using a thiodiglycolic acid as a linking chain. In the reaction, the proportion of the thiodiglycolic acid to the acetic anhydride is 1:1, in mmol: ml; and the molar ratio of the DMAP to the 2-octyl-dodecanol to the thiodiglycolic acid is 0.4:2:1, 2-octyl-dodecanol-thiodiglycolic acid monolateral ester to HOBt to EDCI to DMAP to cabazitaxel is 1:1:2:0.4:0.8.

Mass spectrometry and ¹H-NMR were used to corroborate the structure of the products. The results of the spectral analysis were as follows: ¹H-NMR (600 MHZ, DMSO-d₆) δ7.965 (2H, t), 7.691 (3H, d), 7.349-7.425 (3H, m), 7.152-7.189 (2H, m), 5.753 (1H, d, —NH), 5.352 (1H, d, 3′-H), 5.120 (1H, d, 2-H), 5.026-5.069 (4H, m, 2′-H, —CH=CH—, 5-H), 4.927-4.951 (1H, t, 7-H), 4.682 (1H, d, 20α-H), 4.474 (1H, d, 20β-H), 3.986-4.011 (2H, d, 15α-H, 15β-H), 3.583 (3H, d, 3-H), 3.495 (4H, m, 6α-H, 4-COCH₃), 2.670 (1H, m, 13-H), 2.501 (3H, t, 14α-H, —CH₂CO—), 1.780 (6H, s, —CH₂CH-CHCH₂—), 1.609 (4H, s, 18-H), 1.501 (t, 5H, 6β-H), 1.370 (10H, s, 16-H, 19-H, —CH₂CH₂CO—), 1.210 (24H, t, 17-H), 0.957 (3H, t, —CH₃), 0.831 (5H, t, —CH₂CH₃).

MS (ESI) m/z for C₆₉H₁₀₁NO₁₇SNa [M+Na]⁺: 1271.

### Example 8: Synthesis of Cabazitaxel-2-Octyl-Dodecanol Prodrug Using Monoselenide Bond as Linking Chain

An appropriate quantity of selenodiacetic acid was dissolved in a 25 mL round-bottomed flask containing acetic anhydride, and reacted for 2 h with magnetic stirring. The reaction solution is transferred to a 100 mL round-bottomed flask containing toluene of three times the quantity. The toluene and acetic anhydride in the reaction solution were removed by vacuum rotary evaporation to obtain selenodiacetic anhydride. After mixing selenodiacetic anhydride dissolved in dichloromethane with 2-octyldodecanol, a dichloromethane solution of 4-dimethylaminopyridine (DMAP) was slowly added dropwise to the reaction solution. The reaction was carried out under magnetic stirring for 12 hours to obtain the intermediate product 2-octyldodecyl-selenodiacetic acid monoester. Then, chromatography column method was employed to separate and purify of 2-octyldodecyl-selenodiacetic acid monoester, with cyclohexane-acetone as the mobile phase. The purified previous product, n-(3-dimethylaminopropyl)-n′-ethylcarbodiimide hydrochloride (EDCI), 1-hydroxybenzotriazole (HOBt) and 4-dimethylaminopyridine (DMAP) were dissolved in dichloromethane, activated in an ice bath for 2 h, and then added CTX with stirring at 25° C. for another 48 h. After reaction was completed, the products were separated and purified by the preparative liquid phase, obtaining cabazitaxel-2-octyl-dodecanol prodrug using selenodiacetic acid as a linking chain. In the reaction, the proportion of the monoselenic diacetic acid to the acetic anhydride is 1:1, in mmol: ml; and the molar ratio of the DMAP to the 2-octyl-dodecanol to the selenodiacetic acid is 0.4:2:1, 2-octyl-dodecanol-selenodiacetic acid monolateral ester to HOBt to EDCI to DMAP to cabazitaxel is 1:1:2:0.4:0.8.

Mass spectrometry and ¹H-NMR were used to corroborate the structure of the products. The results of the spectral analysis were as follows: ¹H-NMR (600 MHZ, DMSO-d₆) δ7.969 (2H, t), 7.661 (3H, d), 7.385-7.428 (3H, m), 7.175 (2H, m), 5.807 (1H, d, —NH), 5.355 (1H, d, 3′-H), 5.117 (1H, d, 2-H), 4.932-4.958 (4H, m, 2′-H, —CH=CH—, 5-H), 4.685 (1H, d, 20α-H), 4.474 (1H, d, 20β-H), 3.986-4.011 (2H, d, 15α-H, 15β-H), 3.571-3.829 (3H, d, 3-H) 3.197 (4H, m, 6α-H, 4-COCH₃), 2.670 (1H, m, 13-H), 2.492 (3H, t, 14α-H, —CH₂CO—), 1.795 (6H, s, —CH₂CH=CHCH₂—), 1.501 (4H, s, 18-H), 1.379 (10H, s, 16-H, 19-H, —CH₂CH₂CO—), 1.239 (24H, t, 17-H), 0.958 (3H, t, —CH₃), 0.850 (5H, t, —CH₂CH₃).

MS (ESI) m/z for C₆₉H₁₀₁NO₁₇SeNa [M+Na]⁺: 1319.

### Example 9: Preparation of a Self-Assembled Nanoparticle of the PEG-Modified Small-Molecule Prodrug

0.4 mg of DSPE-PEG_{2K} and 2 mg of prodrug were weighed precisely, and dissolved with 200 μL of acetone, and an ethanol solution was slowly added dropwise into 1.8 mL of deionized water under stirring to spontaneously form a PEG-modified nanoparticle with uniform particle size. Acetone was removed by decompression rotary evaporation to obtain a nanocolloidal solution free of organic reagents. As shown in Table 1, except for a CTX-SS-AA nanoparticle, the nanoparticle has particle size of about 100 nm, particle size distribution of less than 0.2, and surface charge of about −20 mV.

TABLE 1
Particle size, particle size distribution and surface charge
of self-assembled nanoparticle of PEG-modified prodrug

| | Particle | Particle size | Surface charge |
|---|---|---|---|
| Nanoparticle | size (nm) | distribution | (mV) |
| CTX-S-OD nanoparticle | 100.8 | 0.11 | −18.8 |
| CTX-SS-OD nanoparticle | 97.1 | 0.07 | −19.8 |
| CTX-Se-OD nanoparticle | 93.4 | 0.05 | −20.7 |
| CTX-SS-HU nanoparticle | 100.8 | 0.08 | −19.3 |
| CTX-SS-HD nanoparticle | 97.1 | 0.12 | −21.2 |
| CTX-SS-DT nanoparticle | 90.9 | 0.05 | −22.3 |
| γ-CTX-SS-OD nanoparticle | 95.3 | 0.09 | −22.5 |
| CTX-SS-AA nanoparticle | 150.6 | 0.19 | −20.3 |

Results showed that the cabazitaxel prodrug with different linking chains and different fatty alcohol side chains could form the self-assembled nanoparticle. Compared with the self-assembled nanoparticle of the cabazitaxel-straight-chain fatty alcohol small-molecule prodrug, the self-assembled nanoparticle of the cabazitaxel-branched fatty alcohol small-molecule prodrug has smaller particle size and particle size distribution. The particle sizes of the self-assembled nanoparticle of the cabazitaxel-branched fatty alcohol small-molecule prodrug were all about 100 nm, while the particle size distribution is very uniform, with particle size distributions all about 0.1, which is favorable for the nanoparticle to achieve tumor-targeted accumulation through high permeability and retention effect of solid tumors. The surface charge of the nanoparticle is about −20 mV, which is favorable for preventing of aggregation of the nanoparticle by charge repulsion.

### Example 10: Preparation of Lyophilized Powder of the Small Molecule Prodrug Self-Assembled Nanoparticles

1 mL of the PEGylated self-assembled nanoparticles of cabazitaxel-2-octyl-dodecanol prodrug using thioether bond as the linking chain, prepared according to Example 9, was transferred into a penicillin bottle. Various cryoprotectants were used, including 5% sucrose, 10% sucrose, 5% lactose, 10% lactose, 5% trehalose, 10% trehalose, 5% mannitol, 10% mannitol, 5% glucose and 10% glucose. And then the samples were freeze-dried for 24 hours using a lyophilizer, resulting in white powdery cakes. The obtained lyophilized powder was reconstituted with deionized water, and the particle size and surface charge were measured. The results were summarized in Table 2, indicating that using a single cryoprotectant led to relatively larger particle size and broader particle size distribution upon reconstitution.

TABLE 2
Particle size, particle size distribution and surface charge of
lyophilized reconstituted prodrug self-assembled nanoparticles

| | Particle | Particle size | Surface |
|---|---|---|---|
| Nanoparticle | size (nm) | distribution | charge (mV) |
| CTX-S-OD nanoparticle/5% | 281.4 | 0.27 | −22.4 |
| sucrose | | | |
| CTX-S-OD nanoparticle/10% | 113.1 | 0.22 | −19.2 |
| sucrose | | | |
| CTX-S-OD nanoparticle/5% | 379.1 | 0.36 | −24.2 |
| lactose | | | |
| CTX-S-OD nanoparticle/10% | 749.2 | 0.21 | −22.3 |
| lactose | | | |
| CTX-S-OD nanoparticle/5% | 349.6 | 0.3 | −33.6 |
| trehalose | | | |
| CTX-S-OD nanoparticle/10% | 1097 | 0.43 | −23.8 |
| trehalose | | | |
| CTX-S-OD nanoparticle/5% | 691 | 0.37 | −23.1 |
| mannitol | | | |
| CTX-S-OD nanoparticle/10% | 292.8 | 0.34 | −20.9 |
| mannitol | | | |
| CTX-S-OD nanoparticle/5% | 411.8 | 0.41 | −21.3 |
| glucose | | | |
| CTX-S-OD nanoparticle/10% | 592.1 | 0.38 | −19.8 |
| glucose | | | |

### Example 11: Preparation of Lyophilized Powder of the Small Molecule Prodrug Self-Assembled Nanoparticles

1 mL of the PEGylated self-assembled of nanoparticles cabazitaxel-2-octyl-dodecanol prodrug using thioether bond as the linking chain, prepared according to Example 9, was transferred into a penicillin bottle. Various cryoprotectants were used, including 5% lactose and 5% mannitol, 5% lactose and 5% maltose, 5% lactose and 5% sucrose, 5% lactose and 5% trehalose. After ensuring complete dissolution of the cryoprotectants, the samples were pre-frozen at −80° C. for 12 hours, followed by freeze-drying for 24 hours to obtain white powdery cakes. The resulting lyophilized powder was reconstituted with deionized water, and the particle size and the surface charge were measured, with results shown in Table 3. The results showed that the lyophilized powder prepared with composite cryoprotectants had smaller particle sizes upon reconstitution compared to that prepared with single cryoprotectants.

TABLE 3
Particle size, particle size distribution and surface charge of
lyophilized reconstituted prodrug self-assembled nanoparticles

| | Particle | Particle size | Surface |
|---|---|---|---|
| Nanoparticle | size (nm) | distribution | charge (mV) |
| CTX-S-OD nanoparticle | 132.2 | 0.20 | −18.9 |
| 5% lactose + 5% mannitol | | | |
| CTX-S-OD nanoparticle | 161.1 | 0.25 | −20.1 |
| 5% lactose + 5% maltose | | | |
| CTX-S-OD nanoparticle | 143.9 | 0.20 | −22.0 |
| 5% lactose + 5% sucrose | | | |
| CTX-S-OD nanoparticle | 107.5 | 0.21 | −18.4 |
| 5% lactose + 5% trehalose | | | |

### Example 12: Pharmacokinetic Study of Small Molecule Prodrug Self-Assembled Nanoparticles

24 healthy male rats, weighing of 180-220 g, were randomly divided into 4 groups. Prior to administration, the rats were fasted for 12 hours but allowed free access to water. Each group was intravenously administered either cabazitaxel solutions or PEGylated prodrug self-assembled nanoparticle prepared as described in Example 9 at a dose of 4 mg/kg (CTX-equivalent), respectively. Blood samples were collected at designated time points, and drug concentrations in the plasma were measured by liquid chromatography-mass spectrometry tandem. The results were shown in Table 4. The results showed that the cabazitaxel solution had a short circulation time and were rapidly metabolized and cleared from the body after administration. In contrast, the PEGylated prodrug self-assembled nanoparticles showed significantly prolonged circulation times, enhanced bioavailability, and improved pharmacokinetic parameters. Specifically, the overall AUC_{0-24 h} (the sum of both the prodrug and the parent drug) for CTX-SS-OD nanoparticles, CTX-Se-OD nanoparticles and CTX-S-OD nanoparticles was 395.47, 277.37, and 260.19 times higher than that of the cabazitaxel solution, respectively. The half-life (t_{1/2}) was 19.72, 10.16, and 11.51 times longer than that of the cabazitaxel solution, respectively. And the Cₘₐₓ was 76.35, 51.80, and 51.18 times higher than that of the cabazitaxel solutions, respectively. Among these formulations, the CTX-SS-OD nanoparticles demonstrated the most significant improvement in pharmacokinetic parameters, suggesting that disulfide bond, as the linking chain, offered a distinct advantage.

TABLE 4
Pharmacokinetic parameters of cabazitaxel
solution and prodrug nanoparticles

| | | AUC_{0-24 h} | t_{1/2} | Cₘₐₓ |
|---|---|---|---|---|
| Preparation | Determined | (nmol/mL*h) | (h) | (nmol/mL) |
| Cabazitaxel | Cabazitaxel | 1.08 ± | 0.151 ± | 1.7 ± |
| solution | | 0.214 | 0.15 | 0.596 |
| CTX-S-OD | Prodrug | 279.083 ± | 1.739 ± | 85.759 ± |
| | | 86.453 | 0.709 | 7.359 |
| nanoparticle | Cabazitaxel | 1.792 ± | 1.32 ± | 1.248 ± |
| | | 0.631 | 0.256 | 0.356 |
| CTX-Se-OD | Prodrug | 298.679 ± | 1.534 ± | 87.074 ± |
| | | 17.106 | 0.508 | 3.606 |
| nanoparticle | Cabazitaxel | 1.566 ± | 2.462 ± | 0.988 ± |
| | | 0.194 | 1.022 | 0.208 |
| CTX-SS-OD | Prodrug | 420.397 ± | 2.979 ± | 122.48 ± |
| | | 68.584 | 1.398 | 14.47 |
| nanoparticle | Cabazitaxel | 6.718 ± | 1.176 ± | 7.235 ± |
| | | 0.957 | 0.206 | 3.683 |

### Example 13: In Vivo Antitumor Study of PEGylated Small Molecule Prodrug Self-Assembled Nanoparticles

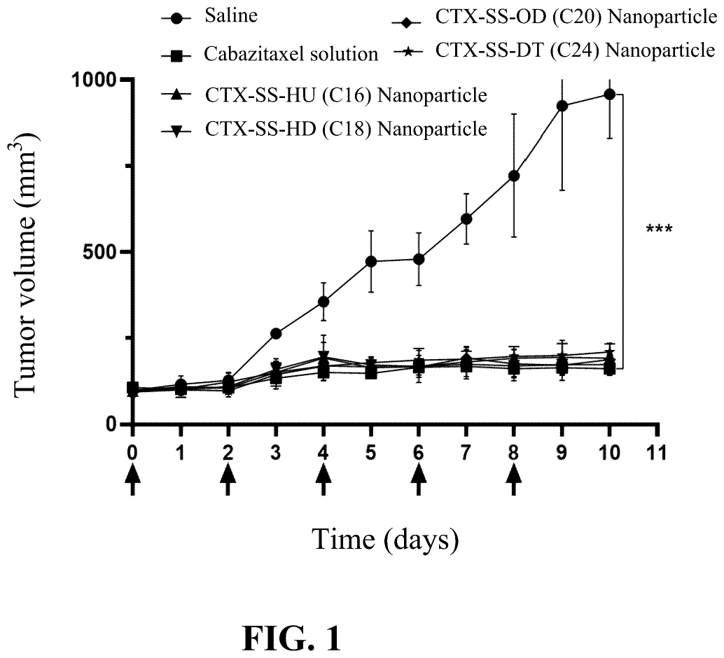

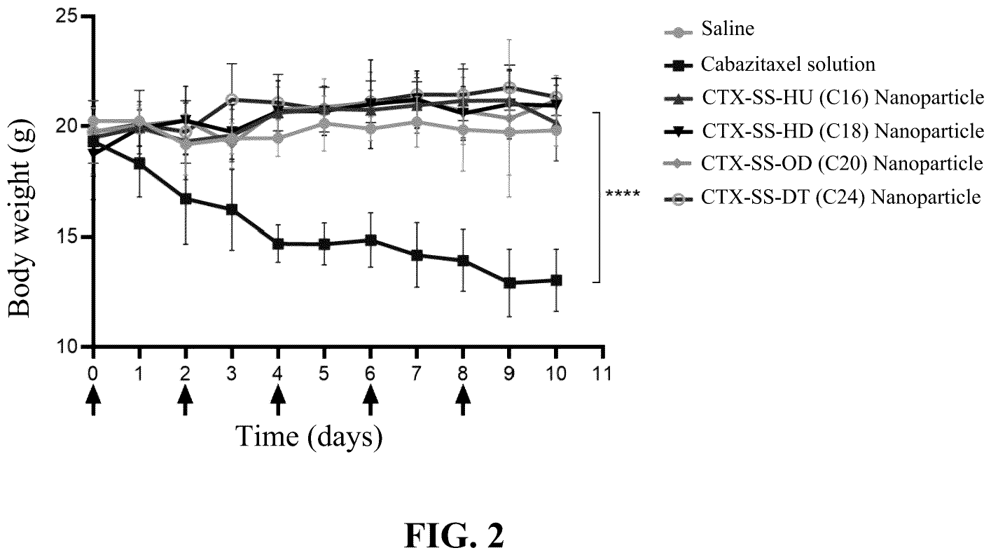

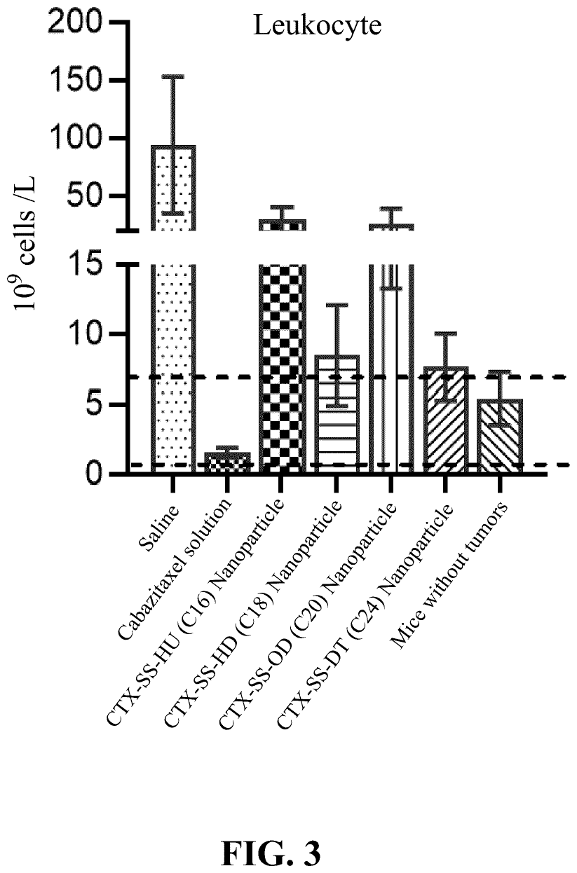

The 4T1 murine breast cancer cell suspension (5×10⁶ cells/100 μL) was inoculated subcutaneously into the dorsal flank of female BALB/c mice. When the tumor volume reached 100-120 mm³, tumor-bearing mice were randomly divided into 6 groups with eight mice per group: Saline, cabazitaxel solution group, CTX-SS-HU nanoparticle group, CTX-SS-HD nanoparticle group, CTX-SS-OD nanoparticle group, and CTX-SS-DT nanoparticle group. The nanoparticles used for treatment were PEGylated prodrug self-assembled nanoparticles, as described in Example 9, with a dosage of 10 mg/kg (CTX equivalent dose). Treatment was administered every other day for a total of five doses. The survival status, body weight, and tumor volume were monitored daily. The mice were euthanized one day after final administration to obtain organs and tumors for further analysis. And the results were shown in FIGS. 1-3. FIG. 1 shows that in the saline group, tumor volume increased rapidly, reaching approximately 1000 mm³ by day 10. In contrast, both the nanoparticles and the cabazitaxel solution significantly inhibited tumor growth (<200 mm³). FIG. 2 indicates that the body weight of mice in the cabazitaxel solution group was significantly decreased, while there was no significant change in the body weight of the prodrug self-assembled nanoparticle group. FIG. 3 illustrates a reduction in white blood cell count in all treatment groups, with the CTX-SS-OD and CTX-SS-HU nanoparticle groups showing the least decrease. In summary, the results suggested that the disulfide bond-linked cabazitaxel-branched fatty alcohol small molecule prodrug self-assembled nanoparticles provide strong antitumor efficacy without inducing significant systemic toxicity, making them a safe and effective chemotherapy drug delivery system.

### Example 14: In Vivo Antitumor Study of PEGylated Small Molecule Prodrug Self-Assembled Nanoparticles

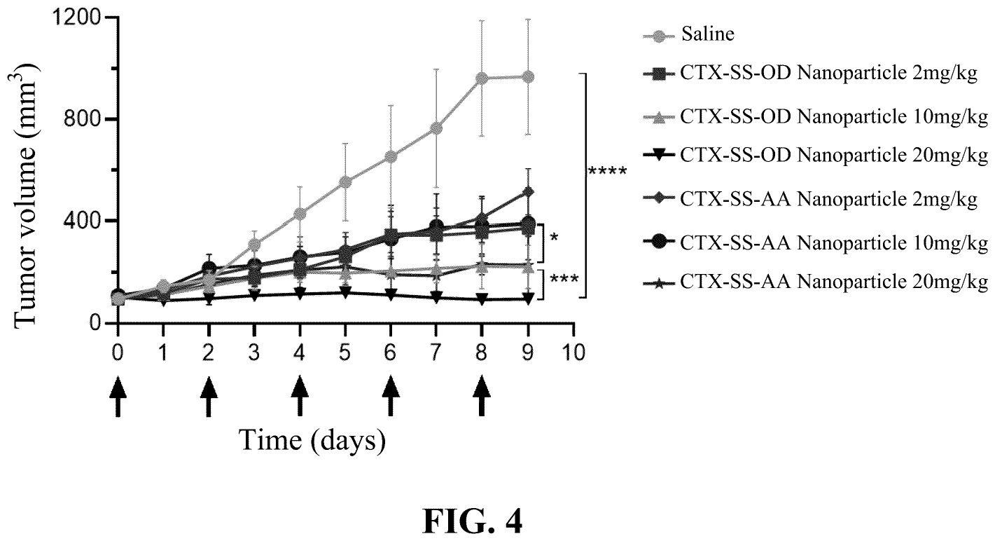

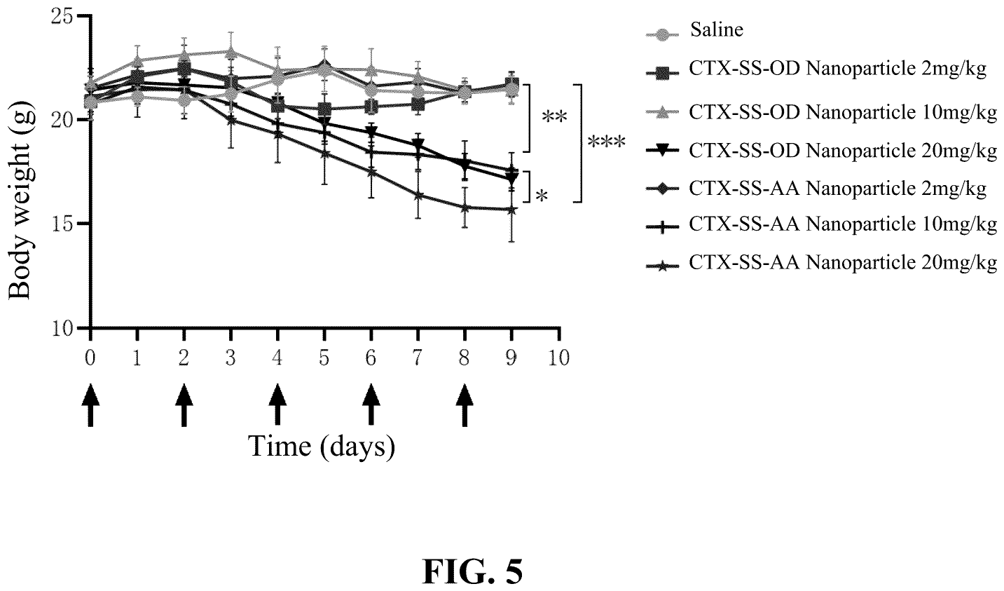

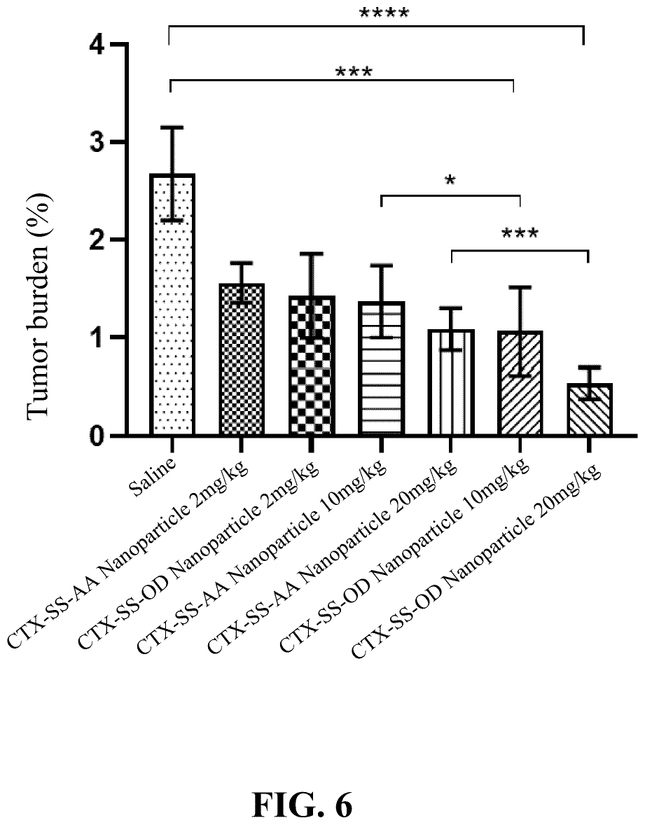

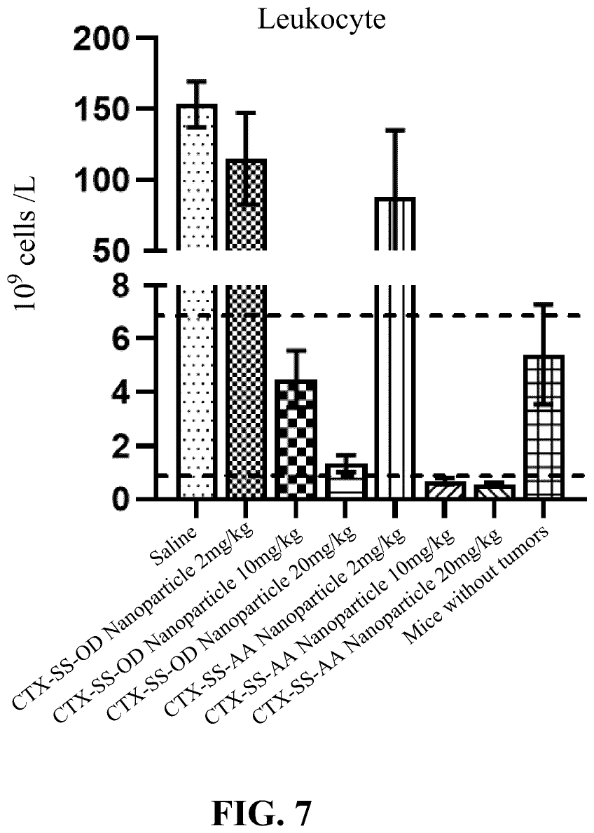

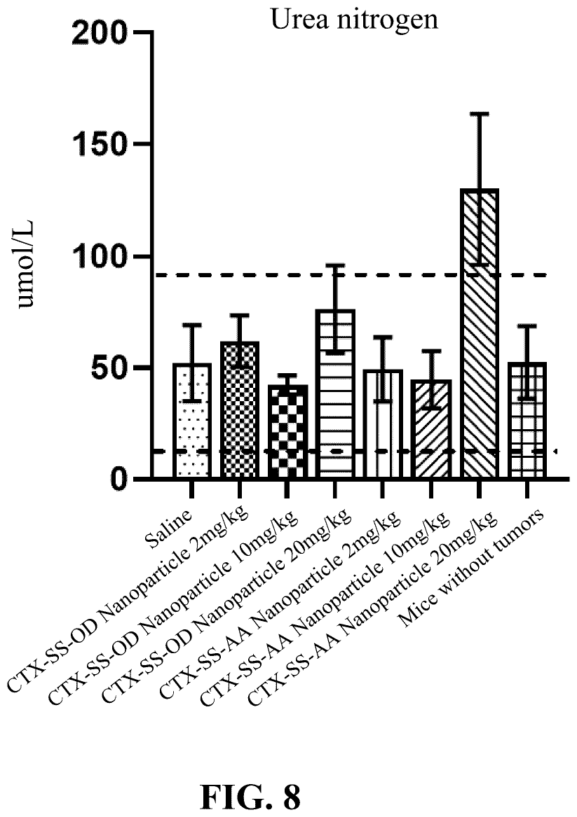

The 4T1 murine breast cancer cell suspension (5×10⁶ cells/100 μL) was inoculated into the dorsal flank of female BALB/c mice. When the tumor volume reached 100-120 mm³, tumor-bearing mice were randomly divided into seven groups, each consisting of 8 mice: saline group, three dosage groups of CTX-SS-OD nanoparticles (2 mg/kg, 10 mg/kg, and 20 mg/kg), and three dosage groups of CTX-SS-AA nanoparticles (2 mg/kg, 10 mg/kg, and 20 mg/kg). The nanoparticles for administration were PEGylated small molecule prodrug self-assembled nanoparticles prepared as described in Example 9, with the dosages calculated based on the equivalent concentration of cabazitaxel. Mice were treated once every other day for a total of five administrations. The survival status, body weight, and tumor volume were monitored daily. The mice were euthanized one day after last administration to obtain organs and tumors for further analysis, and the results were shown in FIGS. 4-8. FIG. 4 showed that in the saline group, tumor volume increased rapidly, reaching approximately 1000 mm³ on day 10, while tumor growth in the prodrug self-assembled nanoparticle groups was significantly inhibited. At the same dose, the CTX-SS-OD nanoparticle group exhibited a smaller tumor volume compared to the CTX-SS-AA nanoparticle group. FIG. 5 showed that, at the same dose, the CTX-SS-AA nanoparticle group experienced a more significant decrease in body weight compared to the CTX-SS-OD nanoparticle group. FIG. 6 illustrates that the prodrug self-assembled nanoparticle groups had a lower tumor burden compared to the saline group, and at the same concentration, the tumor burden in the CTX-SS-OD nanoparticle group was lower than that in the CTX-SS-AA nanoparticle group. FIG. 7 indicated that, at equivalent administration doses, the CTX-SS-OD nanoparticle group showed a smaller reduction in white blood cell count compared to the CTX-SS-AA nanoparticle group. Finally, FIG. 8 reveals that, at a dosage of 20 mg/kg, the CTX-SS-OD nanoparticle group exhibited a smaller increase in urea nitrogen levels compared to the CTX-SS-AA nanoparticle group. These results suggested that the small molecule cabazitaxel-branched fatty alcohol prodrug self-assembled nanoparticles not only exhibit stronger antitumor efficacy but also offer improved safety compared to the small molecule cabazitaxel-straight fatty alcohol prodrug self-assembled nanoparticles.

### Example 15: In Vivo Antitumor Study of PEGylated Small Molecule Prodrug Self-Assembled Nanoparticles

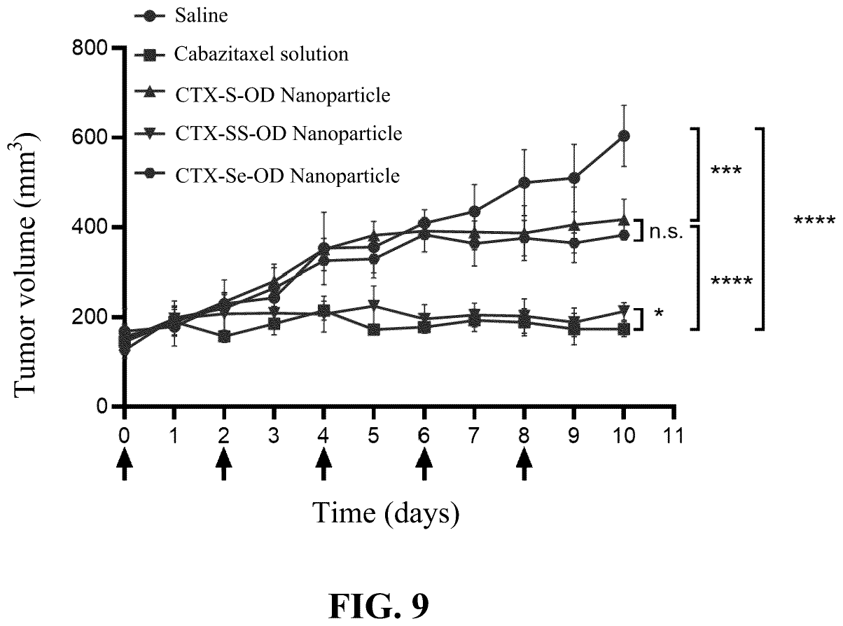

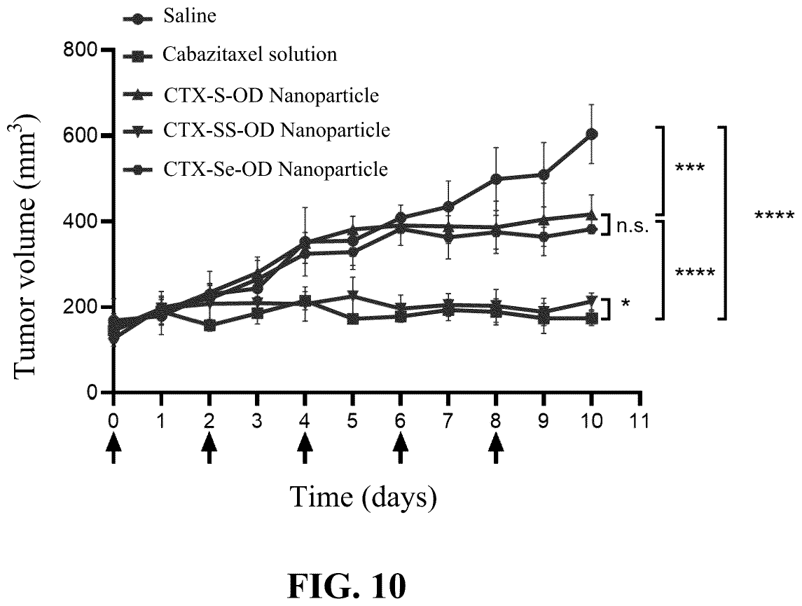

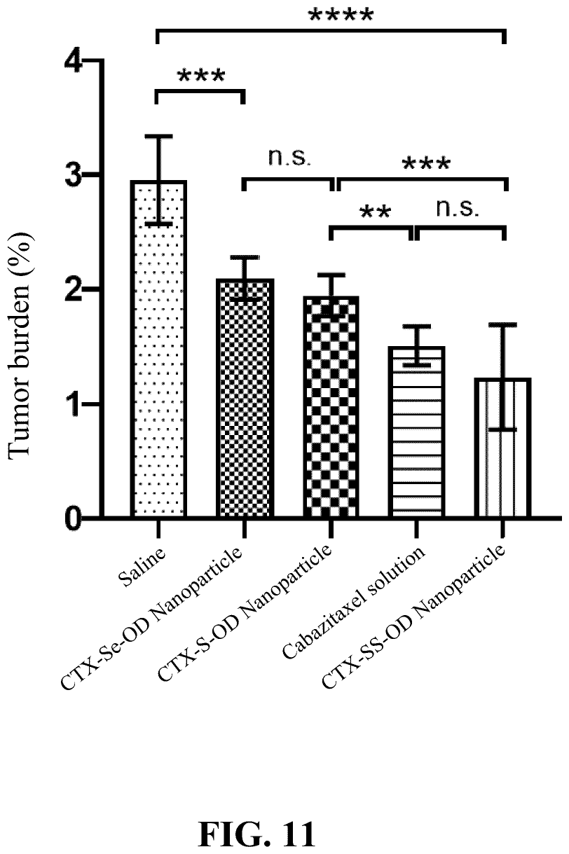

The 4T1 murine breast cancer cell suspension (4T1, 5×10⁶ cells/100 μL) was inoculated into the dorsal flank of female BALB/c mice. Once the tumor volume reached 100-120 mm³, tumor-bearing mice were randomly divided into five groups, each consisting of 8 mice: saline group, cabazitaxel solution group, CTX-S-OD nanoparticle group, CTX-SS-OD nanoparticle group, and CTX-Se-OD nanoparticle group. The nanoparticles administered were PEGylated small molecule prodrug self-assembled nanoparticles, prepared as described in Example 9, with a dosage of 4 mg/kg (cabazitaxel equivalent concentrations). Treatment was administered every other day for a total of 5 doses. The survival status, body weight, and tumor volume were monitored daily. The mice were euthanized one day after final administration to obtain organs and tumors for further analysis, and the results were shown in FIGS. 9-11. FIG. 9 showed that in the saline group, tumor volume increased rapidly, reaching approximately 700 mm³ by day 10. The tumor volumes in the CTX-S-OD nanoparticle group and the CTX-Se-OD nanoparticle group were comparable (<400 mm³), while the tumor volumes in the CTX-SS-OD nanoparticle group and the cabazitaxel solution group were significantly smaller (<200 mm³). FIG. 10 illustrated that there was no significant change in body weight in all three nanoparticle groups, while there was a significant decrease in body weight in the cabazitaxel solution group. FIG. 11 indicated no statistically significant difference in tumor burden between the CTX-SS-OD nanoparticle group and the cabazitaxel solution group. The results suggested that CTX-SS-OD nanoparticles exhibited effective antitumor activity while demonstrating superior safety compared to the cabazitaxel solution. Additionally, the disulfide bond used as the linking chain offered advantages over both thioether and selenoether bonds.

### Example 16: In Vivo Antitumor Study of PEGylated Small Molecule Prodrug Self-Assembled Nanoparticles

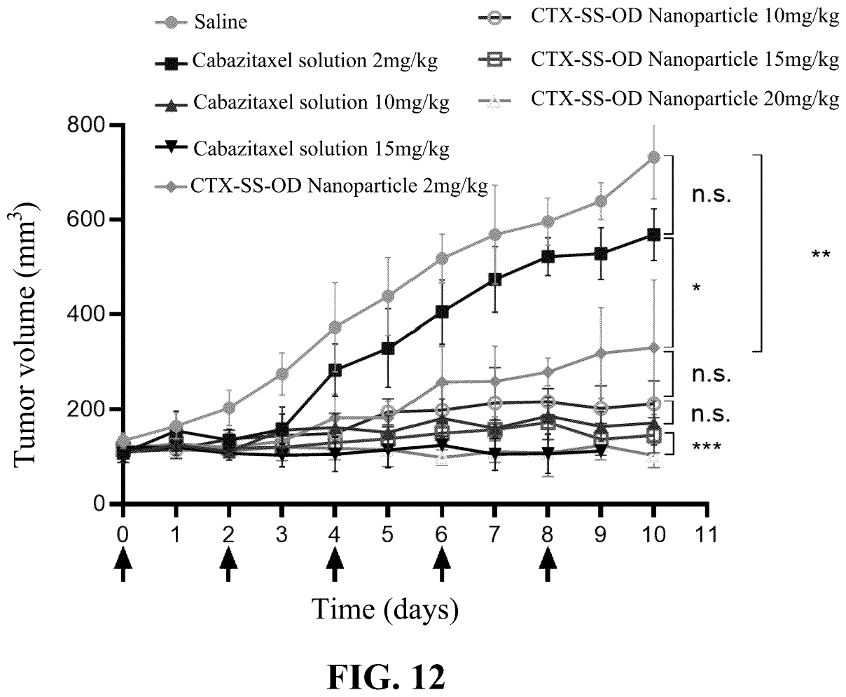

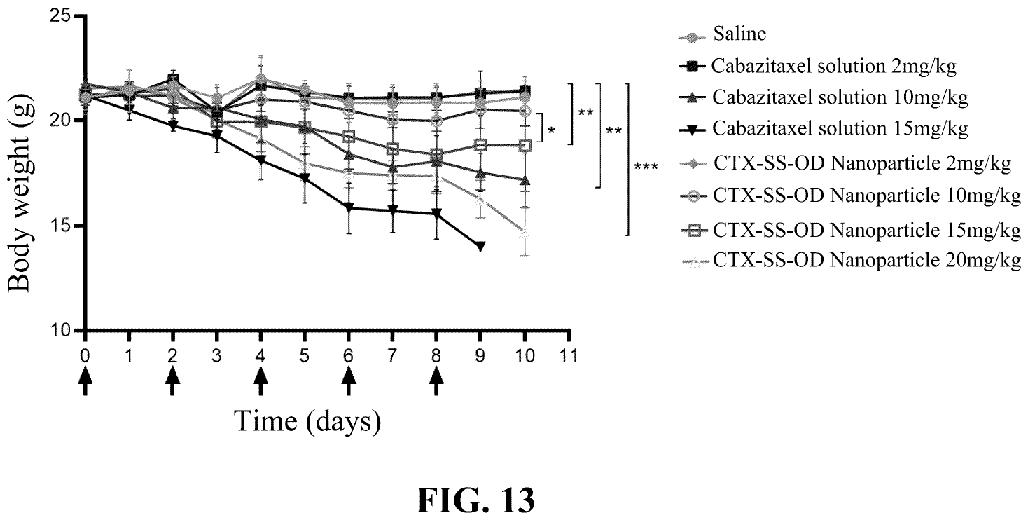

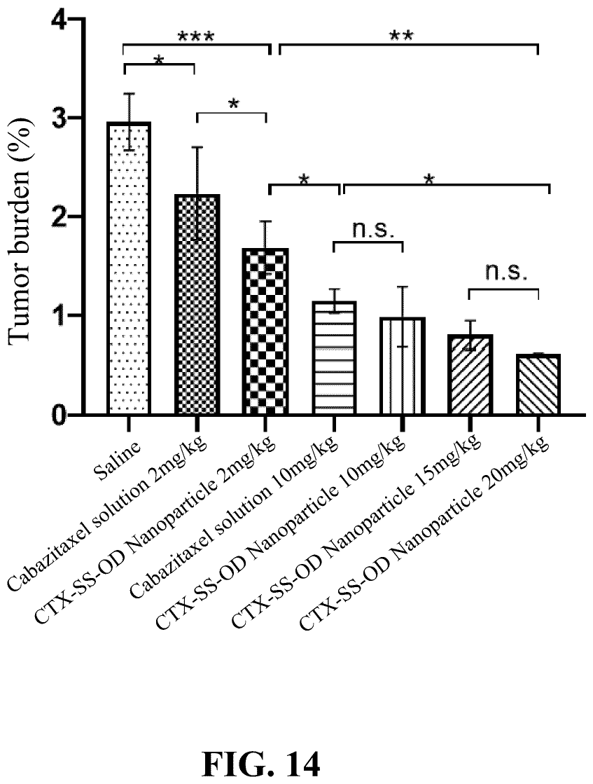

The 4T1 murine breast cancer cell suspension (5×10⁶ cells/100 μL) was inoculated into the dorsal flank of female BALB/c mice. Once the tumor volume reached 100-120 mm³, the tumor-bearing mice were randomly divided into eight, with 8 mice per group: saline group, cabazitaxel solution 2 mg/kg group, cabazitaxel solution 10 mg/kg group, cabazitaxel solution 15 mg/kg group, CTX-SS-OD nanoparticle 2 mg/kg group, CTX-SS-OD nanoparticle 10 mg/kg group, CTX-SS-OD nanoparticle 15 mg/kg group, and CTX-SS-OD nanoparticle 20 mg/kg group. The nanoparticles administered were PEGylated small molecule prodrug self-assembled nanoparticles prepared according to Example 9, and the dosages were calculated at the cabazitaxel equivalent concentrations. Treatment was administered every other day for a total of 5 doses. The survival status, body weight, and tumor volume were monitored daily. The mice were euthanized one day after final administration to obtain organs and tumors for further analysis, and the results were shown in FIGS. 12-14. FIGS. 12 and 14 demonstrated that, in the saline group, tumor volumes increased rapidly and reached approximately 800 mm³ by day 10. At a dose of 2 mg/kg, the tumor volume and the tumor burden in the CTX-SS-OD nanoparticle group were lower than those in the cabazitaxel solution group. At doses of 10 mg/kg and 15 mg/kg, no statistically significant difference in tumor volume or tumor burden was observed between the CTX-SS-OD nanoparticle group and the cabazitaxel solution group. FIG. 13 indicated that, at a dose of 2 mg/kg, there was no significant difference in body weight between the CTX-SS-OD nanoparticle group and the cabazitaxel solution group. At a dose of 10 mg/kg, the CTX-SS-OD nanoparticle group showed no changes in the body weight, while the cabazitaxel solution group exhibited a decrease in the body weight. At the highest dose of 15 mg/kg, all mice in the cabazitaxel solution group died, and no deaths were observed in the CTX-SS-OD nanoparticle group. These results suggested that the CTX-SS-OD nanoparticles exhibited comparable antitumor efficacy to the cabazitaxel solution while demonstrating lower toxicity.

### Example 17: In Vivo Antitumor Study of PEGylated Small Molecule Prodrug Self-Assembled Nanoparticles

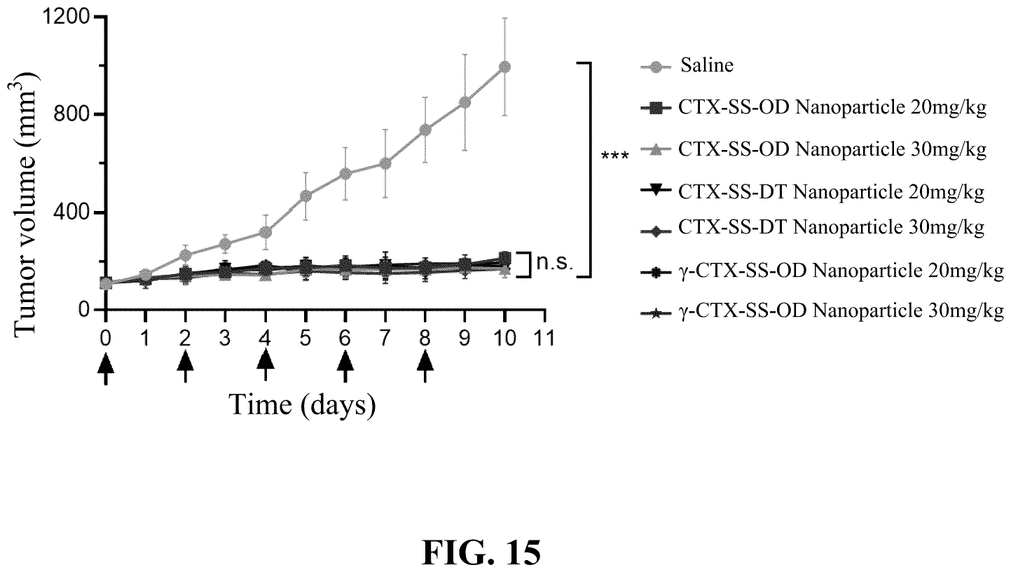

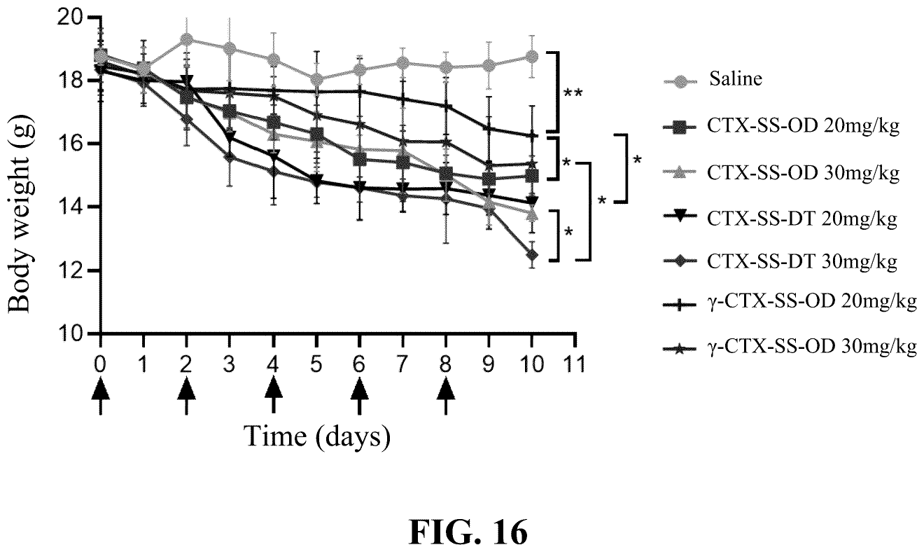

The 4T1 murine breast cancer cell suspension (5×10⁶ cells/100 μL) was inoculated into the dorsal flank of female BALB/c mice. Once the tumor volume reached 100-120 mm³, the tumor-bearing mice were randomly divided into seven groups, with 8 mice per group: saline group, CTX-SS-OD nanoparticle 20 mg/kg group, CTX-SS-OD nanoparticle 30 mg/kg group, CTX-SS-DT nanoparticle 20 mg/kg group, CTX-SS-DT nanoparticle 30 mg/kg group, γ-CTX-SS-OD nanoparticle 20 mg/kg group, and γ-CTX-SS-OD nanoparticle 30 mg/kg group. The nanoparticles used for administration were PEGylated small molecule prodrug self-assembled nanoparticles prepared as described in Example 9, and the administration dose was calculated at the cabazitaxel equivalent concentrations. The treatment was administered every other day for a total of 5 doses. The survival status, body weight, and tumor volume were monitored daily. The mice were euthanized one day after final administration to obtain organs and tumors for further analysis, and the results were shown in FIGS. 15-16. FIG. 15 showed that all three nanoparticles had significant antitumor activity, effectively inhibiting tumor growth. And FIG. 16 demonstrated that the γ-CTX-SS-OD group experienced the least weight loss, suggesting that the γ-CTX-SS-OD nanoparticles have superior safety profiles compared to the CTX-SS-OD and CTX-SS-DT nanoparticle groups.

### Example 18: Tolerance Study of PEGylated Small Molecule Prodrug Self-Assembled Nanoparticles

Female BALB/c mice were divided into 11 groups with 3 mice per group. Four groups received intravenous injections of 30 mg/kg, 40 mg/kg, 50 mg/kg, and 60 mg/kg of cabazitaxel solutions. The remaining seven groups were administered intravenous injections of CTX-SS-HD nanoparticle group, CTX-SS-HU nanoparticle group, CTX-SS-OD nanoparticle group, CTX-SS-DT nanoparticle group, CTX-S-OD nanoparticle group, CTX-SS-AA nanoparticle group, and γ-CTX-SS-OD nanoparticle group every 8 hours, at a dose of 200 mg/kg per administration, with the dosage calculated at the cabazitaxel equivalent concentration, until all mice in the group succumbed. The survival status of the mice was monitored after each administration, and the results were shown in Table 5. The results showed that the prodrug self-assembled nanoparticles exhibited significantly better tolerance than the cabazitaxel solution. Among four nanoparticles, namely the CTX-SS-HD nanoparticle, the CTX-SS-HU nanoparticle, the CTX-SS-OD nanoparticle, and the CTX-SS-DT nanoparticle containing different branched fatty alcohol side chains, the CTX-SS-OD nanoparticles showed the highest safety profile, suggesting that 2-octyl-dodecanol as a side chain offered more safety advantages. When comparing side chains with different structures (branched vs. straight-chain), the CTX-SS-OD nanoparticles, which contain a branched fatty alcohol, demonstrated better safety than the CTX-SS-AA nanoparticles, which contain a straight-chain alcohol. This suggested that branched fatty alcohol was more advantageous than straight-chain fatty alcohols in terms of safety. Further, when comparing nanoparticles with varying lengths of linking chains, γ-CTX-SS-OD nanoparticle containing a longer linking chain exhibited superior safety compared to the CTX-SS-OD nanoparticles. This finding highlights that 4,4′-dithiodibutyric acid as a linking chain conferred greater safety. Among all the nanoparticles, γ-CTX-SS-OD nanoparticle had the highest maximum tolerated dose, which was 40 times higher than that of the cabazitaxel solution.

TABLE 5
Tolerated dose of cabazitaxel solutions and prodrug nanoparticles

| | Maximum | Lowest | Absolute |
|---|---|---|---|
| | tolerated | lethal | lethal |
| Preparation | dose (mg/kg) | dose (mg/kg) | dose (mg/kg) |
| Cabazitaxel solution | 30 | 40 | 60 |
| CTX-SS-HD nanoparticle | 800 | 1000 | 1400 |
| CTX-SS-HU nanoparticle | 800 | 1000 | 1400 |
| CTX-SS-OD nanoparticle | 1000 | 1200 | 1600 |
| CTX-SS-DT nanoparticle | 800 | 1000 | 1600 |
| CTX-S-OD nanoparticle | 1000 | 1200 | 1800 |
| CTX-SS-AA nanoparticle | 600 | 800 | 1200 |
| γ-CTX-SS-OD nanoparticle | 1200 | 1400 | 1800 |

1. (canceled)

2. (canceled)

3. A small molecule cabazitaxel prodrug with branched fatty alcohol or its pharmaceutically acceptable salt, wherein the branched fatty alcohol is one of 2-hexyl-octanol, 1-heptyl-octanol, 2-hexyl-decanol, 1-butyl-dodecanol, 1-heptyl-nonanol, 1-octyl-nonanol, 2-octyl-decanol, 2-heptyl-undecanol, 1-nonyl-decanol, 2-octyl-dodecanol, 2-decyl-tetradecanol or 2-dodecyloctanol.

4. The small molecule cabazitaxel prodrug with branched fatty alcohol or its pharmaceutically acceptable salt according to claim 3, wherein the branched fatty alcohol is 2-hexyl-decanol, 2-heptyl-undecanol, 2-octyl-dodecanol or 2-decyl-tetradecanol; cabazitaxel and branched fatty alcohol in the small molecule cabazitaxel prodrug with branched fatty alcohol are linked by a dibasic acid as a linking chain, and the dibasic acid is monosulfuric dibasic acid, monoselenic dibasic acid, or dithiobasic acid, and the monosulfuric dibasic acid is monosulfuric diacetic acid, monosulfuric dipropionic acid ormonosulfuric dibutyric acid; the monoselenic dibasic acid is monoselenic diacetic acid, monoselenic dipropionic acid or monoselenic dibutyric acid; and the dithiobasic acid is 2,2′-dithiobisacetic acid, 3,3′-dithiodipropionic acid or 4,4′-dithiodibutyric acid.

5. The small molecule cabazitaxel prodrug with branched fatty alcohol or its pharmaceutically acceptable salt according to claim 4, wherein the small molecule cabazitaxel prodrug with branched fatty alcohol or its pharmaceutically acceptable salt is the small molecule cabazitaxel prodrug with branched fatty alcohol or its pharmaceutically acceptable salt adopting the following structure:

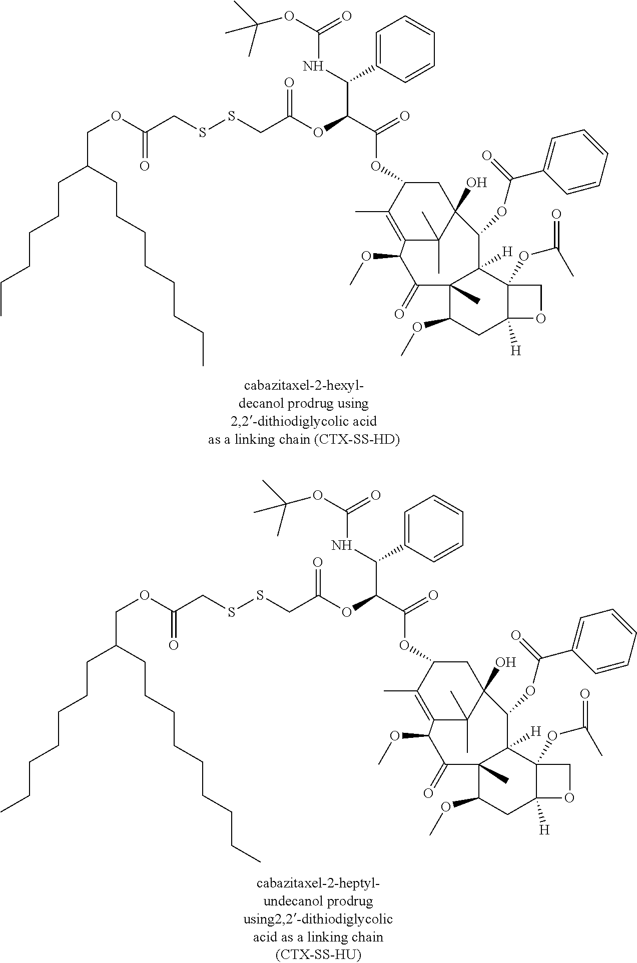

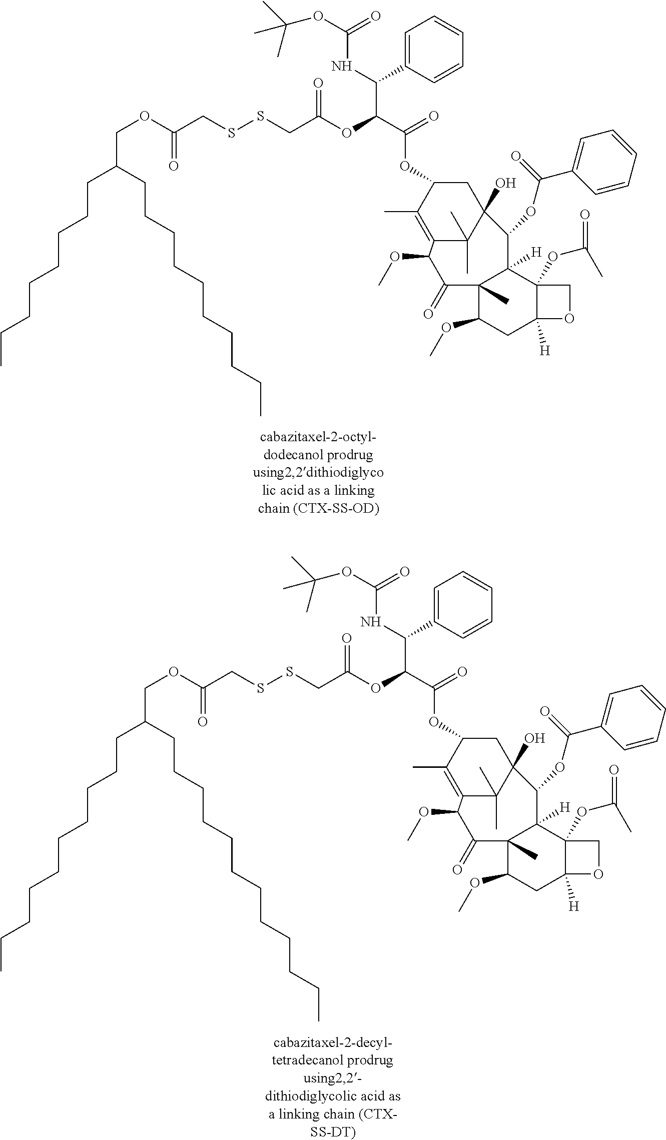

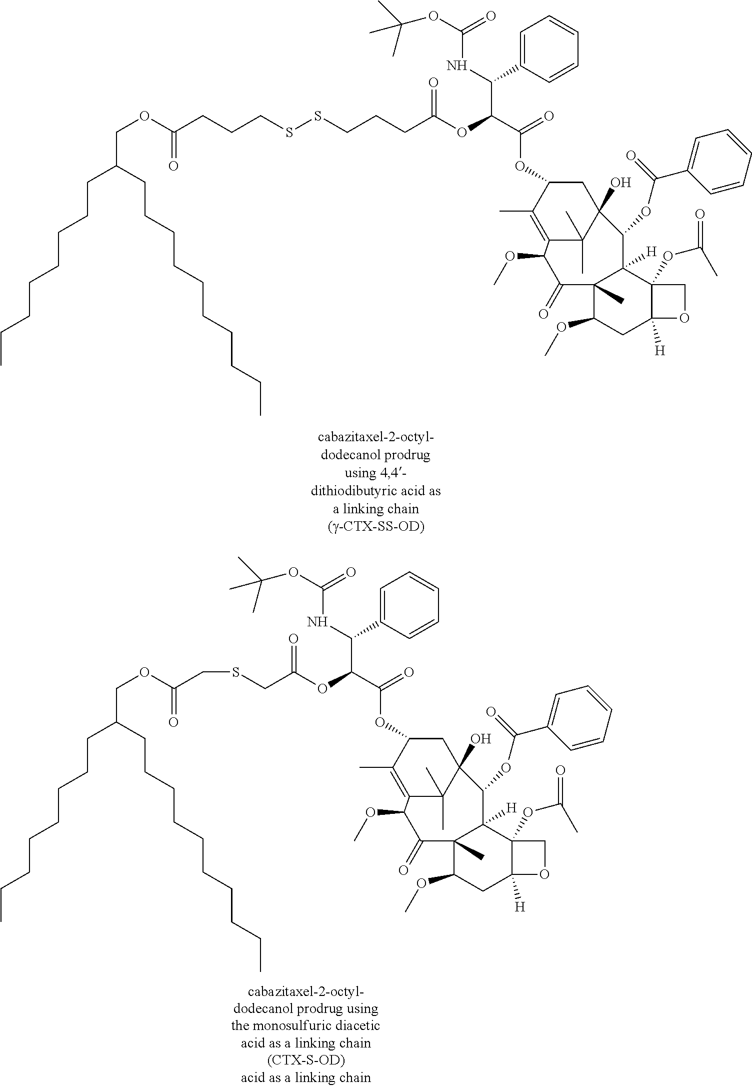

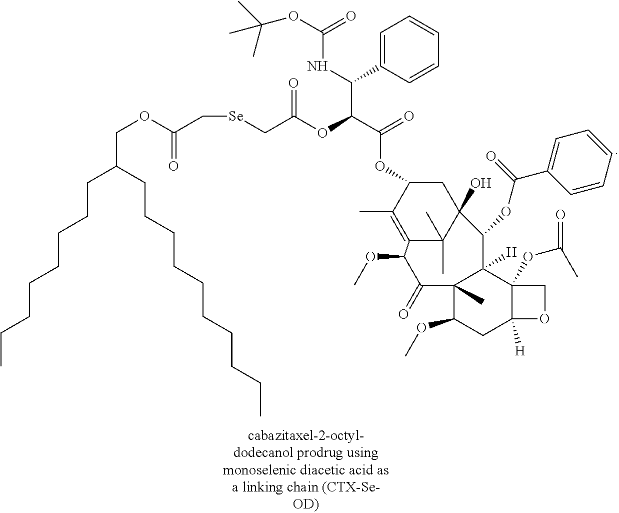

6. The small molecule cabazitaxel prodrug with branched fatty alcohol or its pharmaceutically acceptable salt according to claim 5, wherein synthesis of the small molecule cabazitaxel prodrug with branched fatty alcohol comprises the following steps:
step 1: after dissolving the dibasic acid to obtain dibasic anhydride, performing esterification reaction with the branched fatty alcohol under catalysis of DMAP to obtain an intermediate product, namely a branched fatty alcohol-dibasic acid monolateral ester, wherein a molar ratio of the DMAP to the branched fatty alcohol to the dibasic anhydride is 1: (1-10):(5-15), and the dibasic acid is the monosulfuric dibasic acid, the monoselenic dibasic acid or the dithiobasic acid;
step 2: enabling the branched fatty alcohol-dibasic acid monolateral ester and the cabazitaxel to be subjected to ester-forming reaction to obtain an end product, namely the cabazitaxel-branched fatty alcohol small-molecule prodrug, wherein a molar ratio of the branched fatty alcohol-dibasic acid monolateral ester to the cabazitaxel is 1:(0.5-10) by the following reaction equation: 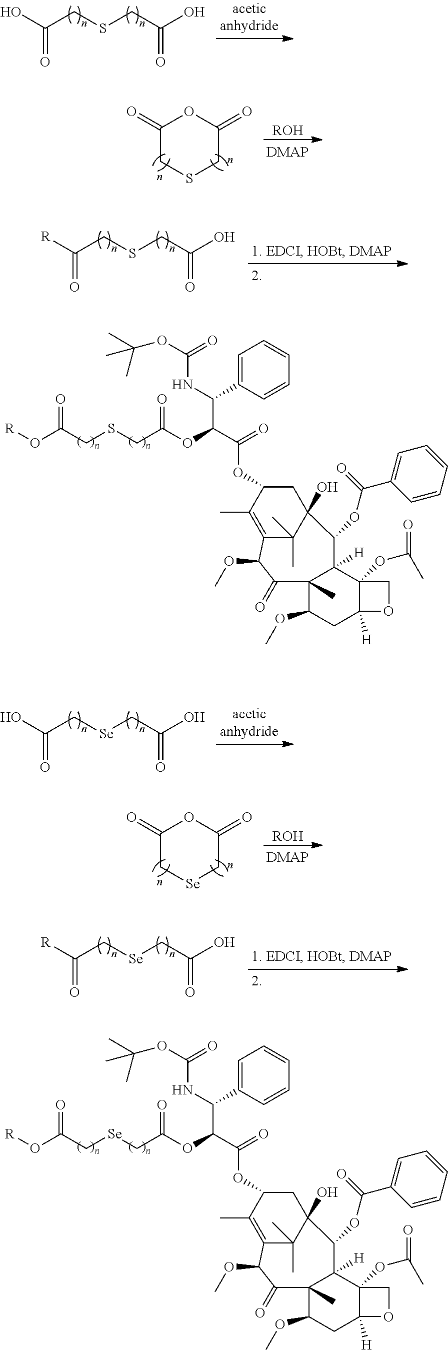 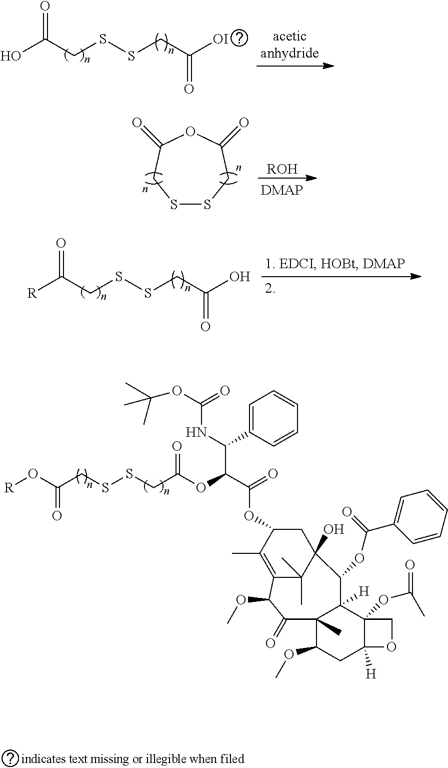
wherein n=1-3, and R is corresponding group of corresponding compound as claimed in claim 4.

7. The small molecule cabazitaxel prodrug with branched fatty alcohol or its pharmaceutically acceptable salt according to claim 3, wherein the small molecule cabazitaxel prodrug with branched fatty alcohol forms a self-assembled nanoparticle, which is a non-PEGylated prodrug-based self-assembled nanoparticle, a PEGylated/active targeting prodrug-based self-assembled nanoparticle or a hydrophobic fluorescent substances/drugs and prodrug co-assembled nanoparticles;
wherein a preparation method of the self-assembled nanoparticle of the small molecule cabazitaxel prodrug with branched fatty alcohol comprises the following steps:
when the self-assembled nanoparticle is the self-assembled nanoparticle of the non-PEGylated small molecule cabazitaxel prodrug with branched fatty alcohol, the preparation method is as follows: dissolve a certain quantity of the prodrug into an appropriate quantity of an organic solvent and dropwise add the solution into water under stirring; the prodrug spontaneously self-assembles into uniform nanoparticle; after removing the organic solvent by vacuum rotary evaporation, a nanocolloidal solution is obtained without any organic solvent, namely the non-PEGylated small molecule cabazitaxel-branched fatty alcohol prodrug-based self-assembled nanoparticles;
when the self-assembled nanoparticle is the self-assembled nanoparticle of small molecule cabazitaxel prodrug with branched fatty alcohol modified by PEG/active targeting groups, the preparation method is as follows: dissolve a certain quantity of the PEG modifier/active targeting modifier and the prodrug in an appropriate quantity of the organic solvent; then, the solvent is slowly dropwise added into water under stirring; the prodrug spontaneously self-assembles into uniform nanoparticles; after removing the organic solvent by vacuum rotary evaporation, the nanocolloidal solution without any organic solvent is obtained, namely the PEGylated/active targeting small molecule cabazitaxel-branched fatty alcohol prodrug-based self-assembled nanoparticles; thereinto, the mass ratio of small molecule cabazitaxel prodrug with branched fatty alcohol to a PEG modifier/active targeting modifier is 1: (0.1-1); the PEG modifier is DSPE-PEG, TPGS, PLGA-PEG, PE-PEG, or DSPE-PEG-FA; the active targeting modifier is antibody, sugar residue, hormone, receptor or ligand;
when the self-assembled nanoparticle is the self-assembled nanoparticle of the small molecule cabazitaxel prodrug with branched fatty alcohol which encapsulates hydrophobic fluorescent substances/drugs, the preparation method is as follows: dissolve a certain quantity of the PEG modifier, the hydrophobic fluorescent substances/drugs, and the small molecule cabazitaxel-branched fatty alcohol prodrug into an appropriate quantity of the organic solvent; then, the solvent isdropwise added into water under stirring; the prodrug spontaneously self-assembles into uniform nanoparticle; after removing the organic solvent by vacuum rotary evaporation, the nanocolloidal solution without any organic solvent is obtained, namely the hydrophobic fluorescent substances/drugs and small molecule cabazitaxel-branched fatty alcohol prodrug co-assembled nanoparticle; thereinto, the mass ratio of the small molecule cabazitaxel prodrug with branched fatty alcohol to the PEG modifier, and the hydrophobic fluorescent substances/drugs is 1: (0.1-1):(0.1-1);
the self-assembled nanoparticle of the small molecule cabazitaxel prodrug with branched fatty alcohol is prepared as a lyophilized powder injection; the lyophilized powder injection includes small molecule cabazitaxel-branched fatty alcohol prodrug self-assembled nanoparticles and lyophilized protective agents; the concentration of prodrug-based self-assembled nanoparticles solution ranges from 0.1 mg/mL-20 mg/mL; the lyophilized protective agent is one or more of glucose, galactose, trehalose, sucrose, mannitol, sorbitol, xylitol, polyethylene glycol, hydroxyethyl starch, or dextran; and a quantity of the lyophilized protective agent ranges from 1%-20% (W/V).

8. (canceled)

9. The small molecule cabazitaxel prodrug with branched fatty alcohol or its pharmaceutically acceptable salt according to claim 3, which is used in the field of anti-tumor drugs, in the field of injection administration, oral administration, topical administration system or in the field of drug delivery system with improving efficacy and reducing toxicity.

10. (canceled)
